(19) United States
(12) Patent Application Publication
(10) Pub. No.: US 2011/0046059 A1
(43) Pub. Date: Feb. 24, 2011

(54) PHARMACEUTICALLY ACCEPTABLE FORMULATIONS/COMPOSITIONS FOR PEPTIDYL DRUGS

(75) Inventors: Gene Scott Merutka, Phoenixville, PA (US); Martin Stogniew, Blue Bell, PA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Zelos Therapeutics, Inc., West Conshohocken, PA (US)

(21) Appl. No.: 12/842,386

(22) Filed: Jul. 23, 2010

Related U.S. Application Data
(63) Continuation-in-part of application No. 12/210,818, filed on Sep. 15, 2008.
Application No. 12/210,818 is a continuation-in-part of application No. 11/890,248, filed on Aug. 2, 2007.
Application No. 11/890,248 is a continuation-in-part of application No. 11/799,816, filed on May 2, 2007.
Application No. 11/799,816 is a continuation-in-part of application No. 11/650,918, filed on Jan. 5, 2007, now abandoned.
Application No. 11/650,918 is a continuation-in-part of application No. 11/517,146, filed on Sep. 6, 2006.
(60) Provisional application No. 61/227,972, filed on Jul. 23, 2009.
(60) Provisional application No. 60/993,884, filed on Sep. 14, 2007.
(60) Provisional application No. 60/925,639, filed on Apr. 20, 2007.
(60) Provisional application No. 60/714,905, filed on Sep. 6, 2005.
(60) Provisional application No. 60/834,980, filed on Jul. 31, 2006.
(60) Provisional application No. 60/837,972, filed on Aug. 15, 2006.

(51) Int. Cl. A61K 38/29; A61P 7/00; A61P 31/00

(52) U.S. Cl. 514/11.8

(57) ABSTRACT

The present invention provides for pharmaceutically acceptable compositions for peptidyl drugs, method of making thereof, and methods of use thereof. Compositions are disclosed comprising a pharmaceutically effective amount of a peptidyl drug and a bioavailability enhancer in an amount sufficient to increase the bioavailability of the peptidyl drug.

### RELATED APPLICATIONS

This application claims the benefit of U.S. Provisional Application No. 61/227,972, filed Jul. 23, 2009, and is a continuation-in part application of U.S. patent application Ser. No. 12/210,818, filed on Sep. 15, 2008, which claims priority to U.S. Provisional Application No. 60/993,884, filed on Sep. 14, 2007, and is a continuation-in-part application of U.S. patent application Ser. No. 11/890,248, filed on Aug. 2, 2007, which claims priority to U.S. Provisional Application No. 60/925,639 filed on Apr. 20, 2007, and is a continuation-in part application of U.S. patent application Ser. No. 11/799,816, filed May 2, 2007, which is a continuation-in part application of U.S. patent application Ser. No. 11/650,918, filed on Jan. 5, 2007, which is a continuation-in-part application of U.S. patent application Ser. No. 11/517,146, filed on Sep. 6, 2006, which claims the benefit of priority to U.S. Provisional Application No. 60/714,905, filed Sep. 6, 2005, and U.S. Provisional Application No. 60/834,980, filed Jul. 31, 2006, U.S. Provisional Application No. 60/837,972, filed Aug. 15, 2006, U.S. Provisional Application No. 60/905,693, filed Mar. 7, 2007, the entire contents of each of which are incorporated herein by reference.

### FIELD OF THE INVENTION

This application relates to pharmaceutical formulations of peptidic drugs.

### BACKGROUND OF THE INVENTION

Use of excipients and cosolvents in parenteral pharmaceuticals is often necessary to overcome challenges in drug solubility of insoluble compounds, typically small molecules. However, commonly used solvents to aid in solubility such as propylene glycol, polyethylene glycol, and glycerol have potential to cause irritation and hemolysis when delivered in large amounts, so their use in parenteral products has been limited. Protein and peptide pharmaceuticals typically have good aqueous solubility, so use of cosolvents in these types of drugs is also limited as the need for solubility enhancers is typically not necessary. What has been unexpectedly observed is that certain cosolvent aqueous mixtures have the potential to increase the relative bioavailability of peptide and protein formulations.

### SUMMARY OF THE INVENTION

The present invention provides for pharmaceutically acceptable formulations/compositions for peptidyl drugs, method of making thereof, and methods of use thereof. According to some embodiments, there is provided a pharmaceutically acceptable parenteral composition comprising: a pharmaceutically effective amount of a peptidyl drug and a bioavailability enhancer in an amount sufficient to increase the bioavailability of the peptidyl drug. According to some embodiments, there is provided a pharmaceutically acceptable subcutaneous composition comprising: a pharmaceutically effective amount of a peptidyl drug and a bioavailability enhancer in an amount sufficient to increase the bioavailability of the peptidyl drug.

The present compositions are useful in methods of administering peptidyl drugs and may be used where it is desirable to increase the blood levels of the peptidyl drug or drugs. The compositions of the present embodiments may assist the user in increasing blood levels of a peptidyl drug by greater than 10% AUC when administered to a subject as compared to a composition that is absent the bioavailability enhancer. In some embodiments, the compositions of the present embodiments may assist the user in increasing blood levels of a peptidyl drug by greater than 20% AUC when administered to a subject as compared to a composition that is absent the bioavailability enhancer.

In some embodiments, the bioavailability enhancer may be present at concentration from about 10% to about 60%. In some embodiments, the bioavailability enhancer is a diol or diol ether. In some embodiments, the diol or diol ether may be between 2 to 20 carbons. In some embodiments, the diol or diol ether is straight-chain or branched, linear or cyclic, aliphatic or aromatic. In some embodiments, the diol or diol ether has a molecular weight of less than 400 MW. In some embodiments, the diol or diol ether has a molecular weight of less than 350 MW. In some embodiments, the diol or diol ether has a molecular weight of less than 300 MW. In some embodiments, the diol or diol ether has a molecular weight of less than 250 MW. In some embodiments, the diol or diol ether has a molecular weight of less than 200 MW. In some embodiments, the diol or diol ether is propylene glycol.

In some embodiments, the composition further comprises a buffer to maintain the composition at a physiological acceptable pH. In some embodiments, the buffer may be present in a concentration of from about 1 mM to about 100 mM.

In some embodiments, the composition further comprises an antioxidant. In some embodiments, the composition further comprises a preservative. In some embodiments, the composition further comprises m-cresol, benzalkonium chloride, parabens, benzyl alcohol, chloroethanol, and mixtures or combinations thereof. In some embodiments, the composition further comprises a bulking/stabilizing agent.

In some embodiments, the composition is formulated for parenteral administration. In some embodiments, the composition is formulated for subcutaneous administration. In some embodiments, the composition is in a sealed sterile glass vial.

According to some embodiments, there is provided a pharmaceutically acceptable subcutaneous composition comprising: a pharmaceutically effective amount of a peptide or peptide mimetic, wherein the peptide or peptide mimetic is less than or equal to 50 amino acids in length; a bioavailability enhancer in an amount sufficient to increase the relative bioavailability of the peptide or peptide mimetic. The bioavailability enhancer may be a diol or diol ether as described herein.

In some embodiments, the peptide or peptide mimetic is 2 to 10 amino acids in length. In some embodiments, the peptide or peptide mimetic is 10 to 50 amino acids in length. In some embodiments, the peptide or peptide mimetic has a molecular weight of less than 8,000 Da. In some embodiments, the peptide or peptide mimetic has a molecular weight of less than 6,000 Da. In some embodiments, the peptide or peptide mimetic has a molecular weight of less than 4,000 Da. In some embodiments, the peptide or peptide mimetic has a molecular weight of less than 2,000 Da.

In some embodiments, the peptidyl drug may be human parathyroid hormone (hPTH), therapeutic fragment thereof, or peptide mimetic thereof. In some embodiments, the peptidyl drug may be a PTH variant, including, but not limited to, the following PTH(1-31), PTH(1-34), PTH(1-37), PTH(1-38), PTH(1-41) and PTH(1-84). In some embodiments, the PTH variant is cyclic.

According to some embodiments, there is provided pharmaceutically acceptable parenteral compositions comprising a pharmaceutically effective amount of a therapeutic protein and a bioavailability enhancer in an amount sufficient to increase the relative bioavailability of said therapeutic protein. In some embodiments, the parenteral compositions are suitable for subcutaneous administration. The bioavailability enhancer may be a diol or diol ether as described herein.

In some embodiments, the one or more therapeutic proteins may be between 8,000 Da and 200,000 Da. In some embodiments, the one or more therapeutic proteins may be between 10,000 Da and 150,000 Da. In some embodiments, the one or more therapeutic proteins may be between 20,000 Da and 150,000 Da. In some embodiments, the protein is an antibody. In some embodiments, the one or more therapeutic proteins may have molecular weight of between 10,000 Da and 20,000 Da. In some embodiments, the one or more therapeutic proteins may have molecular weight of between 20,000 Da and 50,000 Da. In some embodiments, the one or more therapeutic proteins may be 50 to 100 amino acids in length. In some embodiments, the one or more therapeutic proteins may be 100 to 250 amino acids in length.

According to some embodiments, kits are provided comprising a pharmaceutically effective amount of a peptidyl drug in lyophilized form, and a solution for a reconstituting the lyophilized peptidyl drug comprising a bioavailability enhancer in an amount sufficient to increase the bioavailability of the peptidyl drug. The bioavailability enhancer may be a diol or diol ether as described herein.

According to some embodiments, kits are provided comprising a pharmaceutically effective amount of a peptide or peptide mimetic in lyophilized form, wherein the peptide or peptide mimetic is less than or equal to 50 amino acids in length; and a solution for a reconstituting the lyophilized peptide or peptide mimetic comprising a bioavailability enhancer in an amount sufficient to increase the bioavailability of the peptide or peptide mimetic. The bioavailability enhancer may be a diol or diol ether as described herein.

According to some embodiments, kits are provided comprising a pharmaceutically effective amount of a therapeutic protein and a solution for a reconstituting the lyophilized therapeutic protein comprising a bioavailability enhancer in an amount sufficient to increase the bioavailability of the therapeutic protein. The bioavailability enhancer may be a diol or diol ether as described herein.

The present invention provides for methods of preparing the pharmaceutically acceptable formulations/compositions of the present invention.

The present invention provides for methods of treating a subject comprising administering to a subject in need thereof a formulation/composition of the present invention. In some embodiments, the composition is administered subcutaneously.

According to some embodiments, there is provided methods of treating a subject in need thereof comprising administering to the subject the peptidyl drug compositions of the present invention, wherein the amount of bioavailability enhancer is sufficient to reduce the dose given to achieve the same (e.g., within approx. within 10%) pharmacological activity of the peptidyl drug when administered to a subject as compared to a composition that is absent the bioavailability enhancer.

According to some embodiments, there is provided methods of reducing the side effects associated with treating a subject with peptidyl drugs comprising administering to the subject the peptidyl drug compositions of the present invention, wherein the amount of bioavailability enhancer is sufficient to reduce the dose given to achieve the same pharmacological activity of said drug when administered to a subject as compared to a composition that is absent the bioavailability enhancer.

The present invention provides pharmaceutical compositions and formulations containing suitable PTH peptides or analogs thereof for use in methods directed to treating subjects suffering from various bone degenerative, bone deficit, or hematopoietic, or hematological disorders.

In one aspect, the invention provides a method for the treatment of osteoporosis, for treating a bone fracture, for inducing bone formation in trabecular and cortical bones, for treating or preventing renal osteodystrophy (ROD) and related disorders, comprising administering to a subject in need thereof a pharmaceutically acceptable formulation comprising a parathyroid hormone (PTH) peptide or analog, wherein the dosage administered results in an effective pharmacokinetic profile and effective bioactivity.

PTH peptides of the present invention include full-length 1-84, or fragments thereof, including PTH 1-34 and PTH 1-31. Additional PTH peptide analogues can optionally include less than the first 34 amino acids at the N-terminal end.

Specific embodiments of PTH peptide analogues of the present invention include the following: PTH-(1-31)NH₂, Ostabolin; PTH-(1-30)NH₂; PTH-(1-29)NH₂; PTH-(1-28)NH₂; Leu²⁷PTH-(1-31)NH₂; Leu²⁷PTH-(1-30)NH₂; Leu²⁷PTH-(1-29)NH₂; Leu²⁷cyclo(22-26)PTH-(1-31)NH₂ Ostabolin-C™; Leu²⁷cyclo(22-26)PTH-(1-34)NH₂; Leu²⁷cyclo(Lys²⁶-Asp³⁰)PTH-(1-34)NH₂; Cyclo(Lys²⁷-Asp³⁰)PTH-(1-34)NH₂; Leu²⁷cyclo(22-26)PTH-(1-31)NH₂; Ala²⁷ or Nle²⁷ or Tyr²⁷ or Ile²⁷ cyclo(22-26)PTH-(1-31)NH₂; Leu²⁷cyclo(22-26)PTH-(1-32)NH₂; Leu²⁷cyclo(22-26)PTH-(1-31)OH; Leu²⁷cyclo(26-30)PTH-(1-31)NH₂; Cys²²Cys²⁶Leu²⁷cyclo(22-26)PTH-(1-31)NH₂; Cys²²Cys²⁶Leu²⁷cyclo(26-30)PTH-(1-31)NH₂; Cyclo(27-30)PTH-(1-31)NH₂; Leu²⁷cyclo(22-26)PTH-(1-30)NH₂; Cyclo(22-26)PTH-(1-31)NH₂; Cyclo(22-26)PTH-(1-30)NH₂; Leu²⁷cyclo(22-26)PTH-(1-29)NH₂; Leu²⁷cyclo(22-26)PTH-(1-28)NH₂; Glu¹⁷,Leu²⁷cyclo(13-17)(22-26)PTH-(1-28)NH₂; and Glu¹⁷,Leu²⁷cyclo(13-17)(22-26)PTH-(1-31)NH₂.

Other embodiments include any dosage with any route of administration which results in an effective pharmacokinetic profile and effective bioactivity. Administration of the peptidyl or protein formulations of the present invention includes both direct administration, including self-administration, and indirect administration, including the act of prescribing a drug. For example, as used herein, a physician who instructs a patient to self-administer a drug and/or provides a patient with a prescription for a drug is administering the drug to the patient.

A variety of administration routes can be used in accordance with the present invention, including oral, topical, transdermal, nasal, pulmonary, transpercutaneous (wherein the skin has been broken either by mechanical or energy means), rectal, buccal, vaginal, via an implanted reservoir, or parenteral. Parenteral includes subcutaneous, intravenous, intramuscular, intraperitoneal, intra-articular, intra-synovial, intrasternal, intrathecal, intrahepatic, intralesional and intracranial injection or infusion techniques, as well as injectable materials (including polymers) for localized therapy. More preferably, the route of administration is subcutaneous.

The peptidyl drugs of the present invention can also be administered at a variety of doses. Effective dosages can vary according to the type of formulation of peptidyl drug administered as well as the route of administration. One skilled in the art can adjust the dosage by changing the route of administration or formulation, so that the dosage administered would result in a similar pharmacokinetic or biological profile as would result from the preferred dosage ranges described herein.

### BRIEF DESCRIPTION OF THE DRAWINGS

FIG. 1 Mean PTH(1-34) (Teraparatide) Plasma Concentration vs. Time Profiles in Female Sprague Dawley Rats Following Single Subcutaneous 20 μg Fixed Doses Administered as Teraparatide Formulations Containing Either Glycerol or Propylene Glycol or Forteo®.

FIG. 2 Mean PTH(1-34) (Teriparatide) Plasma Concentration vs. Time Profiles in Female Sprague Dawley Rats Following a Single Intravenous 5 μg Fixed Dose of Teriparatide or Following a Single Subcutaneous 20 μg Fixed Dose Administered of Forteo® or Teraparatide Formulations Containing Different Concentrations of Propylene Glycol.

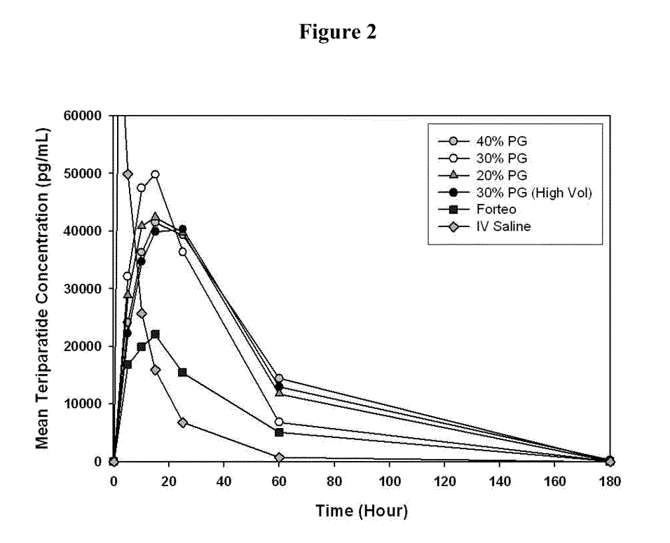

FIG. 3 Mean PTH(1-34) (Teriparatide) Plasma Concentration vs. Time Profiles in Humans Following a Single Intravenous 15 μg Fixed Dose of Teriparatide or Following a Single Subcutaneous 20 μg Dose of Forteo® or Teraparatide Formulations Containing Different Concentrations of Propylene Glycol.

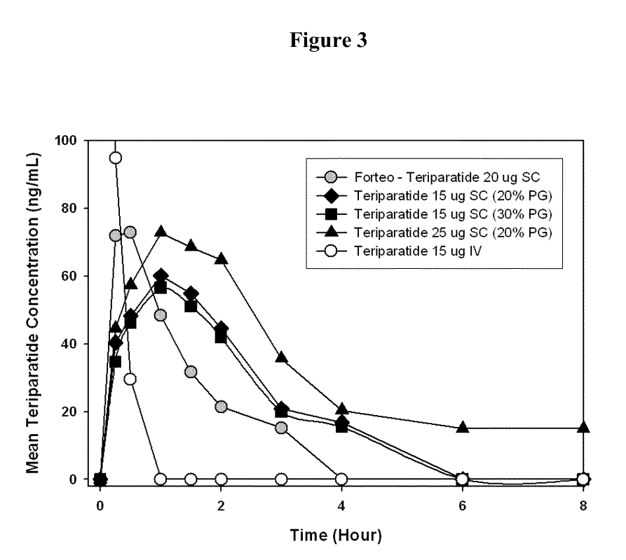

### DETAILED DESCRIPTION OF THE INVENTION

According to some embodiments, the present invention provides for pharmaceutically acceptable formulations/compositions of peptidyl drugs. According to some embodiments, the present invention provides for pharmaceutically acceptable formulations/compositions of therapeutic peptides or peptide mimetics. According to some embodiments, the present invention provides for pharmaceutically acceptable formulations/compositions of therapeutic proteins. The phrase “pharmaceutically acceptable” or “therapeutically acceptable” refers to molecular entities and compositions that are physiologically tolerable and preferably do not typically produce an allergic or similar untoward reaction, such as gastric upset, dizziness and the like, when administered to a human. Preferably, as used herein, the term “pharmaceutically acceptable” means approved by a regulatory agency of the Federal or a State government or listed in the U.S. Pharmacopeia or other generally recognized pharmacopeia (e.g., Remington's Pharmaceutical Sciences, Mack Publishing Co. (A. R. Gennaro edit. 1985)) for use in animals, and more particularly in humans.

A “peptidyl drug” as used herein is an active agent, drug or pharmacologically active agent that comprises a peptide, polypeptide or protein. Pharmacologically active derivatives, variants, mimetics, and fragments of peptidyl drugs are included as well. For ease of discussion, a “peptidyl drug” will also include a single amino acid and derivatives thereof.

A “peptide” refers to a polymer in which the monomers are amino acids linked together through amide bonds. “Peptides” are generally smaller than proteins, i.e., about two to about fifty amino acids in length. The term “peptide” includes “dipeptides” comprised of two amino acids and “tripeptides” comprised of three consecutively linked amino acids, and so forth. The term “polypeptide” is used synonymously with the term “peptide”.

A “protein” as used herein refers to a polymer of amino acids conventionally comprised of over fifty amino acids. The proteins that may be used as peptidyl drugs in the present invention may be naturally occurring proteins, modified naturally occurring proteins, or chemically synthesized proteins that may or may not be identical to naturally occurring proteins.

Diols and Diol Ethers

According to some embodiments, the formulations/compositions of the present invention comprise a pharmaceutically effective amount of one or more peptidyl drugs and a bioavailability enhancer in an amount sufficient to increase the bioavailability of the peptide or peptide mimetic, wherein the bioavailability enhancer is a diol or diol ether.

The diol or diol ether is present in an amount in sufficient to increase the bioavailability of the one or more peptidyl drugs. In some embodiments, the diol or diol ether is present in an amount in sufficient to increase the blood levels of the one or more peptidyl drugs to greater than about 10% (e.g., about 12%, about 15%, about 18%, about 20%, about 22%, about 25%, about 30%, about 50%) AUC when administered to a subject as compared to a formulation without the bioavailability enhancer. In some embodiments, the comparison is made with an identical formulation but where glycerol is used instead of the diol or diol ether.

The composition may be administered subcutaneously, parenterally, intranasally, buccally, intravenously, or trans-mucosally.

According to preferred embodiments, the diol is a diol with 2 to 20 carbons. According to preferred embodiments, the diol is a diol with 2 to 12 carbons (i.e., 2, 3, 4, 5, 6, 7, 8, 9, 10, 11, and 12 carbons). The diols may be straight-chain or branched. The diols may be linear or cyclic. The diols may be aliphatic or aromatic.

According to some embodiments, the diol has a molecular weight of less than about 400 MW (e.g., less than about 350, less than about 300, less than about 250, less than about 200, less than about 150, less than about 100, less than about 50). According to preferred embodiments, the diol has a molecular weight of less than 200 MW.

A representative list of diols useful in the present invention include one or more of following: 1,2-ethanediol; 1,2-propanediol; 1,3-propanediol; 1,2-butanediol; 1,3-butanediol; 2,3-butanediol; 1,4-butanediol; but-2-ene-1,4-diol; 1,2-pentanediol; 1,4-pentanediol, 2,4-Pentanediol, 1,5-pentanediol; 1,2-hexanediol, 1,5-hexanediol, 2,5-hexanediol; 1,6-hexanediol; 1,2-heptanediol; 1,7-heptanediol; 1,2-octanediol; 1,8-octanediol; 1,2-Nonanediol; 1,9-nonanediol; 1,2-decanediol; 1,10-decanediol; 1,2-dodecanediol; 1,12-dodecanediol; 3-methylpentane-1,5-diol; 2,5-dimethyl-1,3-hexanediol; 1,2-cyclopentanediol, 2,2,4-trimethyl-1,3-pentanediol; 1,2-cyclohexanediol; 1,4-cyclohexanediol; 1,4-bis(hydroxymethyl)cyclohexane; neopentyl glycol hydroxypivalate; 2,2-bis(4-hydroxyphenyl)propane; 2,2-bis[4-(2-hydroxypropyl)phenyl]propane; diethylene glycol; triethylene glycol; tetraethylene glycol; dipropylene glycol; tripropylene glycol; tetrapropylene glycol; and 3-thiopentane-1,5-diol, 2,2-dimethyl-1,3-propanediol 2,3-dimethyl-2,3-butanediol; 2,3-dimethyl-1,2-butanediol, 1-phenyl-1,2-propanediol; 2-methyl-1,3-propanediol and mixtures and combinations thereof. According to some embodiments, the diol is propylene glycol.

In some embodiments, diol ethers for use with the present embodiments may have the following structure:

R—(OCH₂CH₂)n-OR′

Where: n=1, 2, or 3; R=alkyl C7 or less or R=phenyl or alkyl substituted phenyl; R′=H or alkyl C7 or less or OR′ is a carboxylic acid ester, sulfate, phosphate, nitrate, or sulfonate.

In some embodiments, diol ethers for use with the present embodiments may have the following structure:

R—[OCH₂CH(CH₃)]n-OR′

where: n=1, 2, or 3; R=alkyl C7 or less or R=phenyl or alkyl substituted phenyl; R′=H or alkyl C7 or less or OR′ is a carboxylic acid ester, sulfate, phosphate, nitrate, or sulfonate.

A representative list of diol ethers useful in the present invention include one or more of following: ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether, diethylene glycol mono-n-butyl ether, propylene glycol methel ether, propylene glycol n-propyl ether, propylene glycol n-butyl ether, propylene glycol phenyl ether, dipropylene glycol methyl ether, dipropylene glycol n-propyl ether, dipropylene glycol n-butyl ether, dipropylene glycol phenyl ether, tripropylene glycol methyl ether, tripropylene glycol n-propyl ether, tripropylene glycol n-butyl ether, and tripropylene glycol phenyl ether.

The diol or diol ether may be present in the formulations/compositions of the present invention in a concentration of from about 10% to about 60% (e.g., about 12%, about 14%, about 16%, about 18%, about 20%, about 22%, about 24%, about 26%, about 28%, about 30%, about 32%, about 34%, about 36%, about 38%, about 40%, about 42%, about 44%, about 46%, about 48%, about 50%, about 60%). This includes, but is not limited to, the following: from about 15% to about 50%; from about 20% to about 50%; from about 25% to about 50%; from about 30% to about 50%; from about 35% to about 50%; from about 40% to about 50%; from about 45% to about 50%; from about 10% to about 40%; from about 15% to about 40%; from about 20% to about 40%; from about 25% to about 40%; from about 30% to about 40%; from about 35% to about 40%; from about 10% to about 30%; from about 15% to about 30%; from about 20% to about 30%; from about 25% to about 30%; and from about 10% to about 20%; and from about 15% to about 20%; from about 50% to about 90%; from about 60% to about 90%; from about 70% to about 90%; from about 50% to about 80%; from about 50% to about 70%; and from about 50% to about 60%.

Peptides

The peptide or peptide mimetic to be used in the formulations/compositions of the present invention include any peptide or peptide mimetic known in the art to have therapeutic qualities. In some embodiments, the peptide is less than or 50 amino acids (e.g., 10, 15, 20, 22, 25, 30, 32, 34, 38, 40, 45, 50). This includes, but is not limited to, less than 50 amino acids, less than 45 amino acids, less than 40 amino acids, less than 35 amino acids, less than 30 amino acids, less than 25 amino acids, less than 20 amino acids, and less than 15 amino acids. For example, the peptide or peptide mimetic may comprise from about 10 to about 50 amino acids; from about 15 to about 50 amino acids; from about 20 to about 50 amino acids; from about 25 to about 50 amino acids; from about 30 to about 50 amino acids; from about 40 to about 50 amino acids; from about 10 to about 40 amino acids; from about 20 to about 40 amino acids; from about 30 to about 40 amino acids; from about 10 to about 35 amino acids; from about 10 to about 30 amino acids; from about 10 to about 25 amino acids; from about 10 to about 20 amino acids; from about 10 to about 15 amino acids; from about 15 to about 35 amino acids; from about 15 to about 55 amino acids; about 20 to about 55 amino acids; about 20 to about 35 amino acids; about 25 to about 35 amino acids; about 2 to about 7 amino acids; about 2 to about 10 amino acids; about 2 to about 15 amino acids; about 2 to about 20 amino acids; about 7 to about 10 amino acids; about 7 to about 15 amino acids; or about 7 to about 20 amino acids.

According to some embodiments, the peptide or peptide mimetic is less than 35 amino acids in length.

According to some embodiments, the peptide or peptide mimetic may less than about 8 kDa. The includes, for example, less than about 8 kDa, less than about 6 kDa, less than about 4 kDa, less than about 2 kDa.

According to some embodiments, the peptide may be PTH (e.g., human PTH) or peptide mimetic thereof. In some embodiments, the peptide mimetic is PTH(1-31), PTH(1-34), PTH(1-37), PTH(1-38), PTH(1-41) and PTH(1-84).

A representative list of peptide drugs useful in the present invention include one or more of following: activin, amylin, angiotensin, atrial natriuretic peptide, calcitonin, calcitonin gene-related peptide, calcitonin N-terminal flanking peptide, cholecystokinin, ciliary neurotrophic factor, corticotropin, corticotropin-releasing factor, epidermal growth factor, follicle-stimulating hormone, gastrin, gastrin inhibitory peptide, gastrin-releasing peptide, ghrelin, glucagon, gonadotropin-releasing factor, growth hormone releasing factor, human chorionic gonadotropin, inhibin A, inhibin B, insulin, leptin, lipotropin, luteinizing hormone, luteinizing hormone-releasing hormone, a-melanocyte-stimulating hormone, (3-melanocyte-stimulating hormone, y-melanocyte-stimulating hormone, melatonin, motilin, oxytocin, pancreatic polypeptide, parathyroid hormone, placental lactogen, prolactin, prolactin-release inhibiting factor, prolactin-releasing factor, secretin, somatotropin, somatostatin, thyrotropin, thyrotropin-releasing factor, thyroxine, triiodothyronine, vasoactive intestinal peptide, vasopressin, buserelin, deslorelin, fertirelin, goserelin, histrelin, leuprolide, lutrelin, nafarelin, triptorelin, abarelix, ghrelin, enterostatin, pentigetide, midkine, magainin, gonadorelin, bradykinin, potentiator B, bradykinin potentiator C, kallidin Cetrotide, DDAVP (desmopressin acetate), and SANDOSTATIN.

Proteins

The therapeutic proteins for use in the formulations/compositions of the present invention include any protein (e.g., antibody) known in the art to have therapeutic qualities. In some embodiments, the therapeutic proteins are between about 8,000 Da and about 200,000 Da, preferably between about 20,000 Da and about 160,000 Da. This includes, for example, proteins having a molecular weight between about 10 kDa and 150 kDa, between about 8 kDa and 20 kDa, between about 10 kDa and 20 kDa, between about 12 kDa and 20 kDa, between about 15 kDa and 20 kDa, between about 20 kDa and 150 kDa, between about 20 kDa and 140 kDa, between about 20 kDa and 120 kDa, between about 20 kDa and 100 kDa, between about 20 kDa and 90 kDa, between about 20 kDa and 80 kDa, between about 20 kDa and 70 kDa, between about 20 kDa and 60 kDa, between about 20 kDa and 50 kDa, between about 20 kDa and 40 kDa, between about 20 kDa and 30 kDa, between about 40 kDa and 150 kDa, between about 40 kDa and 140 kDa, between about 40 kDa and 120 kDa, between about 40 kDa and 100 kDa, between about 40 kDa and 90 kDa, between about 40 kDa and 80 kDa, between about 40 kDa and 70 kDa, between about 40 kDa and 60 kDa, between about 40 kDa and 50 kDa, between about 60 kDa and 150 kDa, between about 60 kDa and 140 kDa, between about 60 kDa and 120 kDa, between about 60 kDa and 100 kDa, between about 60 kDa and 90 kDa, between about 60 kDa and 80 kDa, between about 60 kDa and 70 kDa, between about 80 kDa and 150 kDa, between about 80 kDa and 140 kDa, between about 80 kDa and 120 kDa, between about 80 kDa and 100 kDa, between about 80 kDa and 90 kDa, between about 100 kDa and 150 kDa, between about 100 kDa and 140 kDa, between about 100 kDa and 120 kDa, between about 120 kDa and 150 kDa, and between about 120 kDa and 140 kDa.

A representative list of therapeutic protein or protein drugs useful in the present invention include one or more of following: antithrombin III, factor I, factor II, factor III, factor V, factor VII, factor VIII, factor IX, factor X, factor XI, factor XII, heparin cofactor II, kallikrein, plasmin, plasminogen, prekallikrein, protein C, protein S, thrombo-modulin; colony stimulating factor 4, heparin binding neurotrophic factor, interferon-a, interferon a-2a, interferon a-2b, interferon a-n3, interferon-(3, interferon-y, interleukin-1, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-60 15, interleukin-16, interleukin-17, tumor necrosis factor, tumor necrosis factor-a, granuloycte colony-stimulating factor, granulocyte-macrophage colony-stimulating factor, macrophage colony-stimulating factor, thymopoietin, endorphin, adenosine deaminase, anakinra, ancestim, alteplase, alglucerase, asparaginase, bivalirudin, bombesin, desmopressin acetate, erythropoietin, exendin-4, fibroblast growth factor-2, filgrastim, (3-glucocerebrosidase, hyaluronidase, insulinotropin, lepirudin, nerve growth factor, thrombopoietin, thymosin a-1, thymidin kinase, tissue plasminogen activator, tryptophan hydroxylase, urokinase, urotensin II, RITUXAN, AVASTIN, HERCEPTIN, ERBITUX, TYSABRI, REMICADE, ENBREL, HUMIRA, XOLAIR, and RAPTIVA.

A non-limiting list of peptidyl drugs useful in the present invention are provided in the table below.

| Brand | Generic | Description |
|---|---|---|
| ACTIMMUNE | interferon gamma- | interferon gamma-1b; 140aa; 16.465 KD |
| | 1b | (dimmer of identical monomer of |
| | | 16.465 kD) |
| ARANESP | darbepoetin alfa for | erythropoiesis stimulating protein related |
| | injection | to erythropoietin; 165aa w/5 N- |
| | | glycosylation; 37 kD |
| AVONEX | interferon beta-1a | interferon beta-1a; 166aa glycoprotein; |
| | | 22.5 kD |
| BETASERON | interferon beta-1b | interferon beta-1b; 165aa; not |
| | | glycosylated; 18.5 kD |
| CETROTIDE | cetrorelix acetate | decapeptide with gonadotropin-releasing |
| | for injection | hormone (GnRH) antagonistic activity; |
| | | analogue to GnRH with 5aa substitution; |
| | | 10aa; 1.431 kD |
| DDAVP | desmopressin | analogue of natural pituitary hormone 8- |
| | acetate | arginine vasopressin (ADH), an |
| | | antidiuretic hormone affecting renal water |
| | | conservation; 8aa; 1.183 kD |
| ENBREL | etanercept | dimeric fusion protein consisting of p75 |
| | | TNFR linked to Fc portion of human |
| | | IgG1; 934aa; 150 kD |
| EPOGEN | epoetin alfa | erythropoietin which stimulates red blood |
| | | cell production; 165aa; 30.4 kD; IV & SC |
| GENOTROPIN | somatropin for | recombinant human growth hormone of |
| | injection | pituitary origin (somatropin); 191aa; |
| | | 22.124 kD |
| GLUCAGON | glucagon for | glucagon that increases blood glucose and |
| | injection | relaxes smooth muscle of GI track; 29aa; |
| | | 3.483 kD |
| GONAL-F | follitropin alfa | human follicle-stimulating hormone |
| | injection | (FSH); 92aa & 111aa subunits; 22.69 kD |
| HUMALOG | insulin lispro | human insulin; 21aa & 30aa subunits; |
| | injection | 5.8 kD |
| HUMIRA | adalimumab | human IgG1 monoclonal antibody specific |
| | | for human TNF; TNF blocker; 1330aa; |
| | | 148 kD |
| HUMULIN | huma insulin | human insulin; 21aa & 30aa chains; 5.8 kD |
| | isophane | |
| | suspension | |
| INFERGEN | interferon alfacon-1 | non-naturally occurring type-1 interferon; |
| | | 166aa; 19.43 kD |
| INTRON A | interferon alfa-2b | recombinant alpha interferon product; |
| | | 19.271 kD |
| KINERET | anakinra | Interleukin-1 receptor antagonist (IL-1Ra); |
| | | 153aa; 17.3 kD; |
| LEUKINE | Sargramostim | human granulocyte macrophage colony |
| | | stimulating factor (rhu GM-CSF), |
| | | hematopoietic growth factor; 127aa; |
| | | 19.5 kD, 16.8 kD, & 15.5 kD (glycoprotein |
| | | with 3 primary molecular species) |
| LUVERIS | lutropin alfa for | recombinant human luteinizing hormone |
| | injection | (r-hLH); heterodimer of 92aa & 121aa; |
| | | 29.4 kD |
| NEORECORMON | epoetin beta | human erythropoietin; same primary |
| | | structure as epoetin alfa but different |
| | | glycosylation pattern; 165aa; 30.4 kD |
| NEUBLASTA | pegfilgrastim | covalent conjugate of methionyl human G- |
| | | CSF (filgrastim) and |
| | | monomethoxypolyethylene glycol; 19 kD |
| | | (175aa) filgrastim is bound to 20 kD |
| | | monomethoxypolyethlene glycol to give |
| | | ~39 kD pegfilgrastim |
| NEUMEGA | oprelvekin | interleukin-11, a thrombopoietic growth |
| | | factor; 177aa; 19 kD |
| NEUPOGEN | filgrastim | human granulocyte colony-stimulating |
| | | factor (G-CSF); nonglycosylated; 175aa; |
| | | 18.8 kD; SC & IV |
| NOVOLOG | insulin aspart | insulin analogue; 21aa & 31aa subunits; |
| | | 5.826 kD |
| NUTROPIN | somatropin for | human growth hormone; 191aa; 22.125 kD |
| | injection | |
| OVIDREL | choriogonadotropin | recombinant version of human chorionic |
| | alfa injection | gonadotropin (hCG); 92aa & 145aa |
| | | subunits; 70 kD |
| PEGASYS | peginterferon alfa- | covalent conjugate of recombinant alfa-2a |
| | 2a | interferon (MW 20 kD) with single |
| | | branched bis-monomethoxy polyethlene |
| | | glycol (PEG; MW 40 kD); 60 kD |
| PROCRIT | epoetin alfa | erythropoietin which stimulates red blood |
| | | cell production; 165aa; 30.4 kD |
| RAPTIVA | efalizumab | immunosuppressive recombinant |
| | | humanized IgG1 kappa isotype |
| | | monoclonal antibody that binds to human |
| | | CD11a; 150 kD |
| REBIF | interferon beta-1a | interferon beta- 1a; 166aa; 22.5 kD |
| ROFERON A | interferon alfa-2a | recombinant alpha interferon product; |
| | | 165aa; 19 kD; IM, SC, & IV |
| SAIZEN | somatropin (rDNA | growth hormone; 191aa; 22.125 kD; IM & |
| | origin) for injection | SC |
| SANDOSTATIN | octreotide acetate | cyclic octapeptide that mimic |
| | | pharmacologic actions of hormone |
| | | somatostatin; 8aa; 1.019 kD |
| SEROSTIM | somatropin (rDNA | human growth hormone; 191aa; 22.125 kD |
| | origin) for injection | |
| XOLAIR | omalizumab | recombinant humanized IgG1 k |
| | | monoclonal antibody that selectively binds |
| | | to human IgE; 149 kD |
| ZOLADEX | goserlin acetate | decapeptide analogue of lutenizing |
| | implant | hormone-releasing hormone (LHRH); |
| | | gonadotropin releasing hormone (GnRH) |
| | | agonist analogue; 10aa; 1.269 kD |
| ZORBTIVE | somatropin (rDNA | human growth hormone; 191aa; 22.125 kD |
| | origin) for injection | |

Solubility

According to some embodiments, the active peptidyl drug is at least “very slightly soluble” at 37±1° C. in aqueous media in the pH range of approximately 4-8.0. According to some embodiments, the active peptidyl drug is at least “slightly soluble” at 37±1° C. in aqueous media in the pH range of approximately 4-8.0. According to some embodiments, the active peptidyl drug is at least “sparingly soluble” at 37±1° C. in aqueous media in the pH range of approximately 4-8.0. According to some embodiments, the active peptidyl drug is at least “soluble” at 37±1° C. in aqueous media in the pH range of approximately 4-8.0. In some embodiments, the active peptidyl drug is at least “freely soluble” at 37±1° C. in aqueous media in the pH range of approximately 4-8.0. In some embodiments, the active peptidyl drug is “very soluble” at 37±1° C. in aqueous media in the pH range of approximately 4-8.0.

The USP definitions for “very slightly soluble”, “slightly soluble”, “sparingly soluble”, “soluble”, “freely soluble”, and “very soluble” are provided in the table below.

Values for estimating drug solubility based upon “USP definition”

| Descriptive Term | Parts of Solvent Required for 1 Part Solute |
|---|---|
| Very soluble | Less than 1 |
| Freely Soluble | From 1 to 10 |
| Soluble | From 10 to 30 |
| Sparingly Soluble | From 30 to 100 |
| Slightly Soluble | From 100-1000 |
| Very Slightly | From 1000 to 10,000 |
| Soluble | |
| Insoluble | 10,000 and over |

The United Stated Pharmacopeia, USP 26, NF 21, 2003

Buffers

Any pharmaceutically acceptable buffer may be used in the present formulations/compositions. Preferably, the buffer is present in a concentration of from about 1 mM to about 100 mM (e.g., about 5 mM, about 10 mM, about 15 mM, about 20 mM, about 25 mM, about 30 mM, about 35 mM, about 40 mM, about 50 mM, about 55 mM, about 60 mM, about 65 mM, about 70 mM, about 75 mM, about 80 mM, about 85 mM, about 90 mM, about 95 mM).

The buffer to be included in the formulations may include, but is not limited to, the following: acetate (e.g., sodium acetate), sodium carbonate, citrate (e.g., sodium citrate), tartrate, glycylglycine, histidine, glycine, lysine, arginine, sodium dihydrogen phosphate, disodium hydrogen phosphate, sodium phosphate, and tris(hydroxymethyl)-aminomethan, or mixtures thereof. Each one of these specific buffers constitutes an alternative embodiment of the invention. In a preferred embodiment of the invention the buffer is glycylglycine, sodium dihydrogen phosphate, disodium hydrogen phosphate, sodium phosphate or mixtures thereof.

The buffers are added to maintain the pH of the formulation to between about 3 to about 8 (e.g., about 3, about 3.5, about 4, about 4.5, about 5, about 5.5, about 6, about 6.5, about 6.8, about 7, about 7.2, about 7.3, about 7.4, about 7.5, about 7.6, about 7.7, about 7.8, about 7.9, about 8.0). According to some embodiments, the pH of the formulation may be between about 3 to about 6, between about 3 to about 5.5, between about 3 to about 5.2, between about 3 to about 4.5, between about 3 to about 4, between about 4 to about 5.5, between about 4.5 to about 5.5, between about 4 to about 6, between about 4 to about 7, between about 3 to about 7, between about 4 to about 7.5, between about 5 to about 7.5, between about 5 to about 6.5, between about 5 to about 8, between about 6 to about 8, between about 6.5 to about 7.5, or between about 7.5 to about 8.0). The value of pH assumes normal procedures taken to obtain a pH measurement of 100% aqueous solutions.

Other Additives: Preservatives

Where a pharmaceutically acceptable preservative is to be included in the formulations of the invention, the preservative is selected from the group consisting of phenol, m-cresol, benzalkonium chloride, chloroethanol, methyl p-hydroxybenzoate, propyl p-hydroxybenzoate, parabens, 2-phenoxyethanol, butyl p-hydroxybenzoate, 2-phenylethanol, benzyl alcohol, chlorobutanol, and thiomerosal, or mixtures thereof. Each one of these specific preservatives constitutes an alternative embodiment of the invention. In a preferred embodiment of the invention the preservative is benzyl alcohol, a phenol, or m-cresol.

In a further embodiment of the invention the preservative is present in a concentration from about 0.1 mg/ml to about 50 mg/ml, more preferably in a concentration from about 0.1 mg/ml to about 25 mg/ml, and most preferably in a concentration from about 0.1 mg/ml to about 10 mg/ml.

Administration

For parenteral administration, the compositions of the present invention may be administered by either intravenous, subcutaneous, or intramuscular injection, in compositions with pharmaceutically acceptable vehicles or carriers. The peptidyl drugs may be prepared as compositions/formulations according to the present invention for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles, and can contain formulatory agents, for example, suspending, stabilizing, and/or dispersing agents. Alternatively, the active ingredient can be in powder form for constitution with a suitable vehicle, for example, sterile pyrogen-free water, or sterile cosolvent formulation, before use.

For administration by injection, it is preferred to use the compound(s) in solution in a sterile vehicle which may also contain other solutes such as buffers or preservatives as well as sufficient quantities of pharmaceutically acceptable salts or of glucose to make the solution isotonic. In some embodiments, the pharmaceutical compositions of the present invention may be formulated with a pharmaceutically acceptable carrier to provide sterile solutions or suspensions for injectable administration. In particular, injectables can be prepared in conventional forms, either as liquid solutions or suspensions, solid forms suitable for solution or suspensions in liquid prior to injection or as emulsions. Suitable excipients are, for example, water, saline, dextrose, mannitol, lactose, trehalose, lecithin, albumin, sodium glutamate, cysteine hydrochloride, or the like. In addition, if desired, the injectable pharmaceutical compositions may contain minor amounts of nontoxic auxiliary substances, such as wetting agents, pH buffering agents, and the like.

According to some embodiments, the compositions are in a sealed sterile glass vial. In some embodiments, the compositions are in liquid form. In some embodiments, the compositions are in lyophilized form.

Dosages

Pharmaceutical compositions comprising one or more of proteins, peptides, and mimetics thereof of the present invention may be administered to a patient in an amount sufficient to elicit an effective protective or therapeutic response in the patient. An amount adequate to accomplish this is defined as “therapeutically effective dose.” The dose will be determined by the efficacy of the particular compound employed and the condition of the subject, as well as the body weight or surface area of the area to be treated. The size of the dose also will be determined by the existence, nature, and extent of any adverse effects that accompany the administration of a particular compound or vector in a particular subject.

Exemplary dosages within the scope of the present invention include administering a peptidyl drug in a daily dose selected from about 0.1 mg to about 10 mg (e.g., 0.1, 0.2, 0.3, 0.4, 0.5, 0.6, 0.7, 0.8, 0.9, 1, 2, 3, 4, 5, 6, 7, 8, 9, 10 mg), or fractions thereof, for example 2.5, 4.7, etc. Exemplary dosages for subcutaneous delivery include a daily dose of 2 μg to 10 mg of a formulation stabilized with propylene glycol and/or ethanol, and weekly doses at 3-7 times the daily doses. Other suitable dosages include any dosage with any route of administration that results in a bioavailability or pharmacokinetic profile similar to those yielded by the above-described dosage ranges.

Highly specific peptidyl drugs (e.g., hormones) may be administered at a dose in a daily dose selected from about 1 μg to 200 μg (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 140, 145, 150, 155, 160, 170, 180, 190, or 200 μg), or fractions thereof, for example 12.5, 14.7, etc. Exemplary dosages for subcutaneous delivery include a daily dose of 1 μg to 200 μg of a formulation stabilized with propylene glycol and/or ethanol, and weekly doses at 3-7 times the daily doses. Other suitable dosages include any dosage with any route of administration that results in a bioavailability or pharmacokinetic profile similar to those yielded by the above-described dosage ranges.

Dosages for subcutaneous delivery of an aqueous formulation of peptidyl drugs include dosages between about 0.1 to about 9 mg, about 0.1 to about 8 mg, about 0.1 to about 7 mg, about 0.1 to about 6 mg, about 0.1 to about 5 mg, about 0.1 to about 4 mg, about 0.1 to about 3 mg, about 0.1 to about 2 mg, about 0.1 to about 1 mg, about 0.1 to about 0.5 mg, about 0.1 to about 0.2 mg, 0.2 to about 9 mg, about 0.2 to about 8 mg, about 0.2 to about 7 mg, about 0.2 to about 6 mg, about 0.2 to about 5 mg, about 0.2 to about 4 mg, about 0.2 to about 3 mg, about 0.2 to about 2 mg, about 0.2 to about 1 mg, about 0.2 to about 0.5 mg, 0.5 to about 9 mg, about 0.5 to about 8 mg, about 0.5 to about 7 mg, about 0.5 to about 6 mg, about 0.5 to about 5 mg, about 0.5 to about 4 mg, about 0.5 to about 3 mg, about 0.5 to about 2 mg, or about 0.5 to about 1 mg.

Dosages for subcutaneous delivery of an aqueous formulation of highly specific peptidyl drugs (e.g., hormones) include dosages between 1-9 μg, 10-19 μg, 20-30 μg, 31-40 μg, 42-45 μg, 46-50 μg, and more specifically at 5 μg, 7.5 μg, 10 μg, 15 μg, 20 μg, 25 μg, 30 μg, 35 μg, 40 μg, 45 μg, or 50 μg.

Parathyroid Hormone Analogues

As active ingredient, the pharmaceutically acceptable composition or solution described herein may incorporate full-length PTH (1-84), 1-31 and 1-34 fragments, and other fragments, or variants of fragments, including substitutions, deletions, or insertions, of human PTH, or of rat, porcine or bovine PTH that have human PTH activity as determined in the ovarectomized rat model of osteoporosis reported by Kimmel et al., Endocrinology, 1993, 32(4):1577. The PTH analogues used in the present invention are naturally or non-naturally occurring and may optionally incorporate less than the first 34 N-terminal residues of PTH.

The native PTH sequence, and its truncated 1-34 form, has been shown to have all of these activities. The hPTH-(1-34) sequence is:

(SEQ ID NO: 1)
Ser Val Ser Glu Ile Gln Leu Met His Asn Leu Gly
Lys His Leu Asn Ser Met Glu Arg Val Glu Trp Leu
Arg Lys Lys Leu Gln Asp Val His Asn Phe-OH

Specific embodiments of PTH peptide analogues of the present invention include the following: PTH-(1-34), PTH-(1-34)NH₂, PTH-(1-31)NH₂, Ostabolin; PTH-(1-30)NH₂; PTH-(1-29)NH₂; PTH-(1-28)NH₂; Leu²⁷PTH-(1-31)NH₂; Leu²⁷PTH-(1-30)NH₂; Leu²⁷PTH-(1-29)NH₂; Leu²⁷cyclo(22-26)PTH-(1-31)NH₂ Ostabolin-C™; Leu²⁷cyclo(22-26)PTH-(1-34)NH₂; Leu²⁷cyclo(Lys26-Asp30)PTH-(1-34)NH₂; Cyclo(Lys27-Asp30)PTH-(1-34)NH₂; Leu²⁷cyclo(22-26)PTH-(1-31)NH₂; Ala²⁷ or Nle²⁷ or Tyr²⁷ or Ile²⁷ cyclo(22-26)PTH-(1-31)NH₂; Leu²⁷cyclo(22-26)PTH-(1-32)NH₂; Leu²⁷cyclo(22-26)PTH-(1-31)OH; Leu²⁷cyclo(26-30)PTH-(1-31)NH₂; Cys²²Cys²⁶Leu²⁷cyclo(22-26)PTH-(1-31)NH₂; Cys²²Cys²⁶Leu²⁷cyclo(26-30)PTH-(1-31)NH₂; Cyclo(27-30)PTH-(1-31)NH₂; Leu²⁷cyclo(22-26)PTH-(1-30)NH₂; Cyclo(22-26)PTH-(1-31)NH₂; Cyclo(22-26)PTH-(1-30)NH₂; Leu²⁷cyclo(22-26)PTH-(1-29)NH₂; Leu²⁷cyclo(22-26)PTH-(1-28)NH₂; Glu¹⁷,Leu²⁷cyclo(13-17)(22-26)PTH-(1-28)NH₂; and Glu¹⁷,Leu²⁷cyclo(13-17)(22-26)PTH-(1-31)NH₂.

Exemplary dosages within the scope of the present invention include administering a PTH peptidyl drug in a daily dose selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μg, or fractions thereof, for example 12.5, 14.7, etc. For example, PTH 1-34 may be administered in a daily dose selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 μg, or fractions thereof, for example 12.5, 14.7, etc. PTH 1-84 may be administered in a daily dose selected from 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 μg, or fractions thereof, for example 12.5, 14.7, etc.

The PTH peptidyl drugs of the present invention can also be administered at a variety of doses. Effective dosages can vary according to the type of formulation of PTH peptidyl drug administered as well as the route of administration. One skilled in the art can adjust the dosage by changing the route of administration or formulation, so that the dosage administered would result in a similar pharmacokinetic or biological profile as would result from the preferred dosage ranges described herein.

Exemplary dosages include a daily dose of 2 μg to 50 μg for subcutaneous delivery of a formulation stabilized with propylene glycol and/or ethanol, and weekly doses at 3-7 times the daily doses. Other suitable dosages include any dosage with any route of administration that results in a bioavailability or pharmacokinetic profile similar to those yielded by the above-described dosage ranges.

Preferred dosages for subcutaneous delivery of an aqueous formulation for a PTH peptiyl drug include dosages between 5-9 μg, 10-19 μg, 20-30 μg, 31-40 μg, 42-45 μg, 46-50 μg, and more specifically at 5 μg, 7.5 μg, 10 μg, 15 μg, 20 μg, 25 μg, 30 μg, 35 μg, 40 μg, 45 μg, or 50 μg.

Methods of the Invention and Agents Useful Therein

The methods provided by this invention are generally practiced by administering to an animal in need thereof a daily or weekly dose of a PTH compound in an amount effective to induce bone formation and inhibit or reduce bone loss or resorption.

One aspect of the present invention provides a method for treating osteoporosis by administering to a subject in need thereof a pharmaceutically acceptable formulation comprising a PTH peptide analogue in a daily subcutaneous dose of an aqueous formulation of 2 μg to 100 μg or a weekly dose of from 14 μg to 700 μg, Exemplary dosages include a daily dose of 0.5 to 50 μg for subcutaneous delivery of a formulation stabilized with propylene glycol.

Exemplary dosages within the scope of the present invention include administering PTH 1-34 in a daily dose selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 μg, or fractions thereof, for example 12.5, 14.7, etc., and administering PTH 1-84 in a daily dose selected from 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 μg, or fractions thereof, for example 12.5, 14.7, etc.

Other suitable dosages include any dosage with any route of administration that results in a bioavailability or pharmacokinetic profile similar to those yielded by the above-described dosage ranges. In one embodiment, the subject is a human man or woman. In a preferred embodiment the woman is post-menopausal.

In another embodiment, the osteoporosis can be selected from the group consisting of advanced-stage osteoporosis, hypogonadal osteoporosis, spinal osteoporosis, transplant-induced osteoporosis, and steroid-induced osteoporosis.

Routes of Administration

Administration of the PTH peptide analogues of the present invention includes both direct administration, including self-administration, and indirect administration, including the act of prescribing a drug. For example, as used herein, a physician who instructs a patient to self-administer a drug and/or provides a patient with a prescription for a drug is administering the drug to the patient.

A variety of administration routes can be used in accordance with the present invention, including oral, topical, transdermal, nasal, pulmonary, transpercutaneous (wherein the skin has been broken either by mechanical or energy means), rectal, buccal, vaginal, via an implanted reservoir, or parenteral. Parenteral includes subcutaneous, intravenous, intramuscular, intraperitoneal, intra-articular, intra-synovial, intrasternal, intrathecal, intrahepatic, intralesional and intracranial injection or infusion techniques. More preferably, the route of administration is subcutaneous, transcutaneous, intranasal, transdermal, oral, or inhalation administration.

Formulations

A stabilized solution of a parathyroid hormone can include a stabilizing agent, a buffering agent, a preservative, an antibacterial agent and the like. The stabilizing agent incorporated into the solution or composition includes alcohols, ethanol or a polyol which includes a saccharide, preferably a monosaccharide or disaccharide, e.g., glucose, trehalose, raffinose, or sucrose; a sugar alcohol such as, for example, mannitol, sorbitol or inositol, and a polyhydric alcohol such as glycerine or propylene glycol or mixtures thereof. A preferred polyol is propylene glycol. The concentration of polyol may range from about 10 to about 40 wt-%, preferably about 10-30 wt-% of the total solution.

The buffering agent employed in the solution or composition of the present invention may be any acid or salt combination which is pharmaceutically acceptable. Useful buffering systems are, for example, acetate, tartrate or citrate sources. Preferred buffer systems are acetate or tartrate sources, most preferred is an acetate source. The concentration of buffer may be in the range of about 2 mM to about 500 mM, preferably about 2 mM to 100 mM.

The stabilized solution or composition of the present invention may also include a parenterally acceptable preservative. Such preservatives include, for example, cresols, benzyl alcohol, phenol, benzalkonium chloride, benzethonium chloride, chlorobutanol, phenylethyl alcohol, methyl paraben, propyl paraben, thimerosal and phenylmercuric nitrate and acetate. A preferred preservative is m-cresol or benzyl alcohol; most preferred is m-cresol. The amount of preservative employed may range from about 0.1 to about 2 wt-%, preferably about 0.3 to about 1.0 wt-% of the total solution.

The parathyroid hormone compositions can, if desired, be provided in a powder form containing not more than 2% water by weight, that results from the freeze-drying of a sterile, aqueous hormone solution prepared by mixing the selected parathyroid hormone, a buffering agent and a stabilizing agent as above described. Especially useful as a buffering agent when preparing lyophilized powders is a tartrate source. Particularly useful stabilizing agents include glycine, sucrose, trehalose and raffinose.

Ready to use formulations containing hPTH, or more specifically, Ostabolin-C or PTH(1-34) (Forteo®), are not stable at room temperature and must be stored under refrigerated conditions (2-8° C.). Since hPTH undergoes hydrolysis, oxidation and deamidation in aqueous media, it is difficult to develop a solution formulation for room temperature storage. Although, the formulation is stable at 5° C., it is preferred that the formulation is stable at about pH 7.5, as the pH is closer to physiological pH. Studies have indicated that an Ostabolin-C or PTH(1-34) (Forteo®) solution is less stable at pH 7.5 compared to the ready-to-use formulation. Oxidation and deamidation both occur and takes place above pH 7.0. As such, a 100% aqueous formulation above pH 7 under refrigerated conditions may not be feasible. Hence mixtures of ethanol/water or propylene/water systems were used with the antioxidants methionine or lipoic acid to evaluate the stability of the formulations of this invention.

Another additive to help maintain the stability of an hPTH formulation is Methionine. Methionine has been shown to be a potential antioxidant and improve hPTH stability. Additionally, polyols have the potential to stabilize peptide and protein formulations and sucrose concentrations up to 1M at pH 5.5 have been found to reduce the rate of both deamidation and oxidation of hPTH.

In addition, parathyroid hormones formulated with typical buffers and excipients employed in the art to stabilize and solubilize proteins for parenteral administration. Buffers also have an effect on stability. Previous models showed that for pHs above 7, TRIS buffer had a much lower deamidation rate constant than a corresponding phosphate buffer. Adding NaCl also has a positive effect of the formulation because of its physiological ionic strength. Art recognized pharmaceutical carriers and their formulations are described in Martin, “Remington's Pharmaceutical Sciences,” 15th Ed.; Mack Publishing Co., Easton (1975). More details of stabilizing additives to a hPTH formulation are shown in Examples 15 and 16.

The PTH peptide analogue may also be formulated into a composition suitable for administration by any convenient route, e.g., orally (including sublingually), topically, transdermally (including percutaneous absorption of the composition through the skin, such as by patches, ointments, creams, gels, salves and the like), or intranasally.

Compositions for parenteral administration may be formulated using an injectable liquid carrier such as sterile pyrogen-free water, sterile peroxide-free ethyl oleate, dehydrated alcohol or propylene glycol or a dehydrated alcohol/propylene glycol mixture, and may be injected intravenously, intraperitoneally, subcutaneously or intramuscularly. Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Dosing Regimen

Administration in this invention may consist of one or more cycles; during these cycles one or more periods of osteoclastic and osteoblastic activity will occur, as well as one or more periods when there is neither osteoclastic nor osteoblastic activity. Alternatively, administration may be conducted in an uninterrupted regimen; such a regimen may be a long term regimen, e.g., a permanent regimen.

It will be understood that the dosages of compositions and the duration of administration according to the invention will vary depending on the requirements of the particular subject. The precise dosage regime will be determined by the attending physician or veterinary surgeon who will, inter alia, consider factors such as body weight, age and symptoms (if any). The compositions may if desired incorporate one or more further active ingredients.

During the dosing regimen, the hormone can be administered regularly (e.g., once or more each day or week), intermittently (e.g., irregularly during a day or week), or cyclically (e.g., regularly for a period of days or weeks followed by a period without administration). Regular administration can include once daily, once every two days, once every three days, once every four days, once every five days, once every six days, or once every seven days (once/week). Preferably PTH is administered once daily for 1-7 days for a period ranging from 3 months for up to 3 years in osteoporotic patients. In additional embodiments, PTH is administered for no less than 8 days. The present invention also encompasses embodiments wherein PTH is administered on a weekly basis.

Kits

The present invention also encompasses a kit including the present pharmaceutical compositions and to be used with the methods of the present invention. The kit can contain a vial, for example, which contains a formulation of the present invention and suitable carriers, either dried or in liquid form. The kit further includes instructions in the form of a label on the vial and/or in the form of an insert included in a box in which the vial is packaged, for the use and administration of the compounds. The instructions can also be printed on the box in which the vial is packaged. The instructions contain information such as sufficient dosage and administration information so as to allow a worker in the field to administer the drug. It is anticipated that a worker in the field encompasses any doctor, nurse, or technician who might administer the drug, or a patient who might self-administer the pharmaceutical composition.

In one embodiment the kit contains a medication delivery pen that houses a cartridge assembly containing a vial or cartridge that has the capability of holding about a 60 day supply of daily doses of the pharmaceutical compositions described herein. In additional embodiments, the pen has the capability of holding a 1, 2, 3, 4, 5, 6, 7, or 8 week supply of daily doses of the pharmaceutical compositions described herein. In preferred embodiments, the pen has the capability of holding a 2 or 4 week supply of daily doses of the pharmaceutical compositions described herein. Such a device provides ease of use for self-administration of the pharmaceutical compositions described herein.

In a further embodiment, the cartridge can contain a liquid dosage of the pharmaceutical composition, or a lyophilized dosage, which is reconstituted by the user prior to injection. Those of skill in the pharmaceutical arts will recognize that medication delivery pens, cartridge assemblies for holding a liquid or lyophilized pharmaceutical dosage formulation for same, and methods of lyophilizing and sealing an injectable composition are known in the art, as evidenced by U.S. Pat. Nos. 5,334,162; 6,053,893; and 6,648,859 the teachings of which are incorporated herein by reference.

The examples which follow are illustrative of the invention and are not intended to be limiting.

### Example 1

### Pharmacokinetics of PTH after Intravenous or Subcutaneous Administration of Teriparatide or Forteo® in Female Sprague Dawley Rats

The objective of this study was to evaluate the pharmacokinetics of PTH after single intravenous (IV) or subcutaneous (SC) doses of Teriparatide formulation in propylene glycol cosolvents or Forteo® in female Sprague Dawley rats.

Thirty female Sprague Dawley rats were assigned to six dose groups with five rats per group. Animals in Groups 1 to 5 were administered a single subcutaneous fixed dose of 20 μg PTH(1-34) as teriparatide or as Forteo® (pre-filled pen device) also marketed as FORSTEO in Europe Animals in Group 6 were administered a single intravenous fixed dose of 5 μg teriparatide. Serial blood samples were collected for PTH(1-34) analysis. Summary of the results are provided in Table 1-1.

TABLE 1-1
SUMMARY OF PTH(1-34) PHARMACOKINETICS IN RATS
FOLLOWING SINGLE 20 μG SC FIXED DOSES OF TERIPARATIDE OR
FORTEO ® OR A SINGLE 5 μG IV DOSE OF TERIPARATIDE

| | | Teriparatide | | Teriparatide | | |
|---|---|---|---|---|---|---|
| | | 30% | Teriparatide | 30% | | |
| | Teriparatide | Propylene | 20% | Propylene | | |
| | 40% Propylene | Glycol (Low | Propylene | Glycol (High | | Teriparatide |
| Parameter | Glycol | Volume) | Glycol | Volume) | Forteo ® | in Saline |
| (unit) | Mean | Mean | Mean | Mean | Mean | Mean |
| Cₘₐₓ (pg/mL) | 42775 | 50240 | 43140 | 41420 | 22420 | NA |
| Tₘₐₓ (min) | 20 | 14 | 16 | 23 | 14 | NA |
| t_{1/2} (min) | 18 | 17 | 18 | 21 | 20 | 10 |
| AUCₗₐₛₜ | 2623696 | 2073726 | 2480010 | 2516779 | 976807 | 808540 |
| (min · pg/mL) | | | | | | |
| AUC_{inf} | 2626565 | 2091534 | 2482571 | 2527615 | 1032990 | 819276 |
| (min · pg/mL) | | | | | | |
| CL (mL/min) | NA | NA | NA | NA | NA | 6.268 |
| Vdₛₛ (mL) | NA | NA | NA | NA | NA | 74.008 |
| F_{abs} (%)^{a} | 81 | 64 | 77 | 78 | 30 | NA |
| Fᵣₑₗ (%)^{b} | 269 | 212 | 254 | 258 | NA | NA |
| Body Weight | 324.8 | 311.5 | 325.2 | 309.2 | 313.7 | 312.3 |
| (g) | | | | | | |

^{a}Absolute bioavailability relative to the IV dose, where % F_{abs} = [(AUC_{last SC} × Dose_{IV})/(mean AUC_{last IV} × Dose_{SC})] × 100
^{b}Bioavailability relative to the Forteo ® dose, where % Fᵣₑₗ = (AUC_{last Teriparatide}/mean AUC_{last Forteo}) × 100
NA: Not Applicable

All animals in the study were dosed successfully. There were no abnormalities noted at the time of dosing or during sample collection with the exception of one rat in Group 4 with moderate to severe erythema. There was no erythema or edema formation in any of the other animals post-dose. In each group, plasma PTH(1-34) concentrations were quantifiable at least to 60 minutes post-dose. Comparing the mean pharmacokinetic parameters, exposure to PTH(1-34) following IV and SC dosing, measured as Cₘₐₓ and AUC, was higher following SC administration of Teriparatide compared to SC dosing with Forteo®. Mean absolute bioavailability (% F_{abs}) of PTH(1-34) (compared to IV Teriparatide administration) following SC dosing was 81% with a 40% propylene glycol formulation, 64% using a 30% propylene glycol, low volume formulation (20 μL), 77% for a 20% propylene glycol formulation, 78% with a 30% propylene glycol, high volume formulation (50 μL), and 30% for Forteo®. Mean relative bioavailability (% Fᵣₑₗ) of PTH(1-34), compared to Forteo® SC administration, was 269% for the 40% propylene glycol formulation, 212% for the 30% propylene glycol, low volume formulation (20 μL), 254% for the 20% propylene glycol formulation, and 258% for the 30% propylene glycol, high volume formulation (50 μL). The mean terminal half-life was similar for each SC dose group, ranging from 17 to 21 minutes. The mean terminal half-life following IV dosing was 10 minutes.
This section provides abbreviations used throughout this report.
AUCₗₐₛₜ Area under the plasma concentration-time curve from time zero to the time of the last measurable concentration
AUC_{inf} Area under the plasma concentration-time curve from time zero to infinity
CL Systemic clearance
Cₘₐₓ Maximum plasma concentration
F_{abs} Absolute bioavailability
FVC Femoral vein cannula(ted)
Fᵣₑₗ Relative bioavailability
Forteo® teriparatide (rDNA origin) injection
IV Intravenous
JVC Jugular vein cannula(ted)
NA Not Applicable
SC Subcutaneous
t_{1/2} Terminal half-life
Tₘₐₓ Time of maximum plasma concentration
Vdₛₛ Volume of distribution at steady state

The PTH(1-34) AUCₗₐₛₜ following SC dosing of teriparatide or Forteo® compared to PTH(1-34) AUCₗₐₛₜ following IV dosing with teriparatide resulted in mean absolute bioavailability values (F_{abs}) of 81% for teriparatide in the 40% propylene glycol formulation, 64% for teriparatide in the 30% propylene glycol formulation (low dosing volume), 77% for teriparatide in the 20% propylene glycol formulation, 78% for teriparatide in the 30% propylene glycol formulation (high dosing volume), and 30% for Forteo®.

The PTH(1-34) AUCₗₐₛₜ following SC dosing of teriparatide compared to PTH(1-34) AUCₗₐₛₜ following SC dosing with Forteo® resulted in mean relative bioavailability values (Fᵣₑₗ) of 269% for teriparatide in the 40% propylene glycol formulation, 212% for teriparatide in the 30% propylene glycol formulation (low dosing volume), 254% for teriparatide in the 20% propylene glycol formulation, and 258% for teriparatide in the 30% propylene glycol formulation (high dosing volume).

Terminal half-life (t_{1/2}) was similar for all five SC dose groups. The mean t_{1/2} was 18 minutes following teriparatide administration in the 40% propylene glycol formulation, 17 minutes following teriparatide administration in the 30% propylene glycol formulation (low dosing volume), 18 minutes following teriparatide administration in the 20% propylene glycol formulation, 21 minutes following teriparatide administration in the 30% propylene glycol formulation (high dosing volume), and 20 minutes following Forteo® administration. Mean t_{1/2} following IV administration of 5 μg teriparatide in saline was 10 minutes.

Dosing Formulations: Formulations for IV and SC dosing were prepared on the day of dosing as described below.

Group 1 Formulation—Teriparatide (1 mg/mL adjusted for 90.8% peptide assay, 1.1 mg/mL) in 40% propylene glycol, 0.7% NaCl, 50 mM Tris, 5 mg/mL methionine, 3 mg/mL m-cresol solvent buffer: 9.4 mg of teriparatide was weighed out and combined with 8.54 mL of 40% propylene glycol, 0.7% NaCl, 50 mM Tris, 5 mg/mL methionine, 3 mg/mL m-cresol solvent buffer, Lot BA041409-05. The formulation was mixed for 5 minutes and vortexed for 1 minute after which the pH was determined to be 7.38. No pH adjustment was made. The total formulation weight was 8.9786 g.

Group 2 Formulation—teriparatide (1 mg/mL adjusted for 90.8% peptide assay) in 30% propylene glycol, 0.7% NaCl, 50 mM Tris, 5 mg/mL methionine, 3 mg/mL m-cresol solvent buffer: 9.3 mg of teriparatide was weighed out and combined with 8.44 mL of 30% propylene glycol, 0.7% NaCl, 50 mM Tris, 5 mg/mL methionine, 3 mg/mL m-cresol solvent buffer, Lot BA041409-06. The formulation was mixed for 5 minutes and vortexed for 1 minute after which the pH was determined to be 7.63. No pH adjustment was made. The total formulation weight was 8.7815 g.

Group 3 Formulation—Teriparatide (1 mg/mL adjusted for 90.8% peptide assay) in 20% propylene glycol, 0.7% NaCl, 50 mM Tris, 5 mg/mL methionine, 3 mg/mL m-cresol solvent buffer: 8.7 mg of teriparatide was weighed out and combined with 7.90 mL of 20% propylene glycol, 0.7% NaCl, 50 mM Tris, 5 mg/mL methionine, 3 mg/mL m-cresol solvent buffer. The formulation was mixed for 5 minutes and vortexed for 1 minute after which the pH was determined to be 7.55. No pH adjustment was made. The total formulation weight was 8.1944 g.

Group 4 Formulation—Teriparatide (0.4 mg/mL adjusted for 90.8% peptide assay) in 30% propylene glycol, 0.7% NaCl, 50 mM Tris, 5 mg/mL methionine, 3 mg/mL m-cresol solvent buffer: to 4 mL of the 30% propylene glycol formulation used for Group 2 dosing (Lot #BA041509-02 above) was added 6 mL 30% propylene glycol, 0.7% NaCl, 50 mM Tris, 5 mg/mL methionine, 3 mg/mL m-cresol solvent buffer. The formulation was mixed for 5 minutes after which the pH was determined to be 7.61. No pH adjustment was made. The total formulation weight was 10.3073 g.

Group 5 Formulation—Forteo® (0.25 mg/mL solution): the Forteo® solution was removed directly from the pre-filled pen device into the dosing syringe using another needle to vent the device.

Group 6 Formulation—Teriparatide (0.25 mg/mL adjusted for 90.8% peptide assay) in 0.9% NaCl (sterile saline): 5.3 mg of teriparatide was weighed out and combined with 19.25 mL of saline, mixed for 5 minutes and vortexed for 1 minute after which the pH was determined to be 5.5. No pH adjustment was made. The total formulation weight was 19.3410 g. The formulation was clear and assigned.

Dose Administration Thirty female Sprague Dawley rats (25 JVC, 5 FVC/JVC) were received was and there was a minimum of 2 days acclimation prior to releasing all animals to this study. Animals were assigned to six dose groups with 5 rats per group. Animals in Groups 1 to 5 were administered a single SC fixed dose of teriparatide or Forteo® at 20 μg/animal Animals in Group 6 were administered a single IV fixed dose of teriparatide at 5 μg/animal via FVC. The dose groups and doses are as follows:

| | | | | | Dose | Dose | |
|---|---|---|---|---|---|---|---|
| | # of | Test | | Dose | Level | Conc. | Dose Vol. |
| Group | Animals | Article | Formulation^{a} | Route | (μg) | (mg/mL) | (μL/animal) |
| 1 | 5 | Terapartide | 40% propylene | SC | 20 | 1 | 20 |
| | | | glycol | | | | |
| 2 | 5 | Terapartide | 30% propylene | SC | 20 | 1 | 20 |
| | | | glycol | | | | |
| 3 | 5 | Terapartide | 20% propylene | SC | 20 | 1 | 20 |
| | | | glycol | | | | |
| 4 | 5 | Terapartide | 30% propylene | SC | 20 | 0.4 | 50 |
| | | | glycol | | | | |
| 5 | 5 | Forteo ® | solution | SC | 20 | 0.25 | 80 |
| 6 | 5 | Terapartide | saline | IV | 5 | 0.25 | 20 |

Sample Collections: Blood samples were collected from each animal in Groups 1-5 at pre-dose and at 5, 10, 15, 25, 60 and 180 min post-dose (0.5 mL). Blood samples were collected from each animal in Group 6 at pre-dose and at 2, 5, 10, 15, 25, 60 and 180 min post-dose (0.3 mL for samples collected pre-dose through 10 minutes post-dose, 0.5 mL for samples collected after 10 minutes post-dose). Non-terminal blood samples were collected via the JVC, and the terminal (180 min) blood sample was collected by cardiac puncture. Blood was collected into labeled tubes containing K₂EDTA. Plasma samples were stored at approximately −70° C. until analysis.

Pharmacokinetic Analysis: Individual plasma concentration-time data were used to determine the pharmacokinetic parameters. Non-compartmental pharmacokinetic parameters were calculated using the WinNonlin™, version 4.1 (WinNonlin™ Copyright© 2003, Pharsight Corporation, Mountain View, Calif.). The terminal elimination rate constant (λ) was obtained, where possible, by linear regression of the terminal elimination phase of a log-linear plot of the plasma concentration-time data. The apparent terminal-phase elimination half-life (t_{1/2}) was calculated according to the following formula:

t_{1/2}=^{ln(2)}/_{λ}

Acceptance criteria for λ determination required regression of at least three points from the elimination phase and r² equal to or greater than 0.85.

The area under the plasma concentration-time curve from time 0 to the time of the last measurable plasma concentration (AUCₗₐₛₜ) was determined by the linear trapezoidal method. AUC_{inf} was calculated as:

AUC_{inf}=AUCₗₐₛₜ+Cₗₐₛₜ/λ

Nominal sampling times were used for t_{1/2} and AUC calculations, since samples were taken at the scheduled times.

Systemic clearance (CL) and volume of distribution at steady-state (Vdₛₛ) following IV administration were calculated according to the following:

$$CL = \frac{Dose}{{AUC}_{inf}} {Vd}_{ss} = {MRT}_{inf} \cdot CL$$

Bioavailability was calculated using AUCₗₐₛₜ. Absolute bioavailability (% F_{abs}) for individual rats in the SC dose groups was calculated relative to the IV dose group, calculated as:

% F_{abs}=[(AUC_{last SC}×Dose_{IV})/(mean AUC_{last IV}×Dose_{SC})]×100

The bioavailability for individual rats in the teriparatide dose groups relative to the Forteo® dose group (% Fᵣₑₗ) was calculated as:

% Fᵣₑₗ=(AUC_{last teriparatide SC}/mean AUC_{last Forteo SC})×100

Nominal doses were used to calculate CL, Vdₛₛ and F.

### Example 2

### Pharmacokinetics of PTH after Subcutaneous Administration of Three Formulations of Teriparatide or Forteo® in Female Sprague Dawley Rats

The objective of this study was to evaluate the pharmacokinetics of PTH after single subcutaneous (SC) doses of TERIPARATIDE or Forteo® in female Sprague Dawley rats.

Twenty female Sprague Dawley rats were assigned to four dose groups with five rats per group. All animals were administered a single fixed subcutaneous dose of 20 μg as teriparatide (3 formulations) or as Forteo® (teriparatide injection, Eli Lilly and Co.). Serial blood samples were collected for PTH(1-34) analysis.

All animals on study were dosed successfully. There were no abnormalities noted at the time of dosing or during sample collection. There was no erythema or edema formation at the injection was observed for any animal. In each group, plasma PTH(1-34) concentrations were quantifiable at least to 60 minutes post-dose. Exposure to PTH(1-34) following SC dosing, measured by mean Cₘₐₓ and AUC, was greater following administration of teriparatide compared to Forteo®. Mean relative F of PTH(1-34) following teriparatide dosing was 143% for the 50% glycerol low volume formulation, 112% for the 50% glycerol high volume formulation, and 294% for the 40% propylene glycol formulation. The mean terminal half-life ranged from 19 to 23 minutes in the 4 groups. Group mean (±SD) pharmacokinetics are summarized below.

TABLE 2-1
Summary of PTH(1-34) Pharmacokinetics in Rats Following Single 20 μg
Subcutaneous Injection Doses of teriparatide (Three Formulations) or Forteo ®

| | Teriparatide | Teriparatide | Teriparatide | |
|---|---|---|---|---|
| | 50% Glycerol | 50% Glycerol | 40% Propylene | |
| | (Low Volume) | (High Volume) | Glycol | Forteo ® |
| Parameter (unit) | Mean | Mean | Mean | Mean |
| Cₘₐₓ (pg/mL) | 23440 | 16180 | 32120 | 16960 |
| Tₘₐₓ (min) | 14 | 13 | 21 | 13 |
| t_{1/2} (min) | 19 | 23 | 22 | 21 |
| AUCₗₐₛₜ | 1107367 | 850556 | 2291939 | 748614 |
| (min · pg/mL) | | | | |
| AUC_{inf} (min · pg/mL) | 1118442 | 870975 | 2302104 | 783432 |
| Relative F (%)^{a} | 143 | 112 | 294 | NA |
| Body Weight (g) | 325.8 | 324.9 | 325.9 | 324.5 |

^{a}Bioavailability (F) relative to the Forteo dose, where F(%) = AUC_{inf teriparatide}/mean AUC_{inf Forteo} × 100
NA: Not Applicable

Formulations for SC dosing were prepared on the day of dosing as described below.

Group 1 Formulation—1 mg/mL teriparatide in 50% glycerol (v/v), 0.7% NaCl, 50 mM Tris, 5 mg/mL Methionine buffer: 10.46 mg of teriparatide was weighed and combined with 9.509 mL of 50% Glycerol in Tris aqueous buffer stock. The formulation was mixed on a magnetic stirrer for 5 minutes after which the pH was determined to be 7.5. No pH adjustment was made. The formulation was clear and colorless and was assigned. The nominal concentration of teriparatide was 1.0 mg/mL after adjusting for 90.8% peptide assay.

Group 2 Formulation—0.4 mg/mL teriparatide in 50% glycerol, 0.7% NaCl, 50 mM Tris, 5 mg/mL Methionine buffer: 4 mL of Group 1 Formulation was combined with 6 mL of 50% Glycerol in Tris aqueous buffer stock. The formulation was mixed on a magnetic stirrer for 5 minutes, and the pH was determined to be 7.6. No pH adjustment was made. The formulation was clear and colorless. The nominal concentration of teriparatide was 0.4 mg/mL after adjusting for 90.8% peptide assay.

Group 3 Formulation—1 mg/mL teriparatide in 40% propylene glycol, 0.7% NaCl, 50 mM Tris, 5 mg/mL Methionine buffer: 7.92 mg of teriparatide was weighed and combined with 7.2 mL of 40% Propylene Glycol in Tris aqueous buffer stock. The formulation was mixed on a magnetic stirrer for 5 minutes, after which the pH was determined to be 7.6. No pH adjustment was made. The formulation was clear and colorless.

Group 4 Formulation—Forteo® (0.25 mg/mL solution): The Forteo® solution was removed directly from the pre-filled pen into the dosing syringe using a second needle to vent the chamber.

### Results:

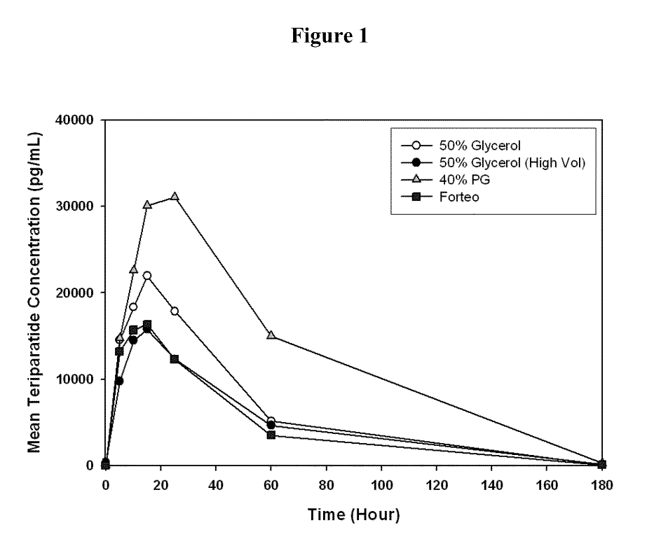

Mean PTH(1-34) plasma concentration versus time profiles for Groups 1 through 4 are shown in FIG. 1.

In each group, plasma PTH(1-34) concentrations were quantifiable at least to 60 minutes post-dose. Exposure to PTH(1-34) following a single fixed 20 μg SC dose, measured by mean Cₘₐₓ and mean AUC, was greater following administration of teriparatide in three different formulations compared to Forteo®. Of the teriparatide dose groups (Groups 1-3), exposure to PTH(1-34) was greatest when teriparatide was administered in the 40% propylene glycol formulation.

Mean Cₘₐₓ was 23440 pg/mL following administration of teriparatide formulated in 50% glycerol (low dosing volume), 16180 pg/mL following administration of teriparatide formulated in 50% glycerol (high dosing volume), 32120 pg/mL following administration of teriparatide formulated in 40% propylene glycol, and 16960 pg/mL following administration of Forteo®.

Similarly, mean AUC_{inf} was 1118442 pg·min/mL following administration of teriparatide formulated in 50% glycerol (low dosing volume), 870975 pg·min/mL following administration of teriparatide formulated in 50% glycerol (high dosing volume), 2302104 pg·min/mL following administration of teriparatide formulated in 40% propylene glycol, and 783432 pg·min/mL following administration of Forteo®.

The PTH(1-34) AUC_{inf} following SC dosing of teriparatide compared to AUC_{inf} following SC dosing with Forteo® resulted in mean relative bioavailability values of 143% for teriparatide in the 50% glycerol formulation (low dosing volume), 112% for teriparatide in the 50% glycerol formulation (high dosing volume) and 294% for teriparatide in the 40% propylene glycol formulation.

The terminal half-life (t_{1/2}) was similar for all four dose groups. The mean terminal half-life was 19 minutes following teriparatide administration in the 50% glycerol formulation (low dosing volume), 23 minutes following teriparatide administration in the 50% glycerol formulation (high dosing volume), 22 minutes following teriparatide administration in the 40% propylene glycol formulation, and 21 minutes following administration of Forteo®.

TABLE 2-2
Dosing Groups and Doses

| | | | | | Dose | Dose | |
|---|---|---|---|---|---|---|---|
| | # of | Test | | Dose | Level | Conc. | Dose Vol. |
| Group | Animals | Article | Formulation | Route | (μg) | (mg/mL) | (μL/animal) |
| 1 | 5 | teriparatide | 50% glycerol | SC | 20 | 1 | 20 |
| 2 | 5 | teriparatide | 50% glycerol | SC | 20 | 0.4 | 50 |
| 3 | 5 | teriparatide | 40% | SC | 20 | 1 | 20 |
| | | | propylene | | | | |
| | | | glycol | | | | |
| 4 | 5 | Forteo ® | solution | SC | 20 | 0.25 | 80 |

### Example 3

### Subcutaneous Formulations of PTH

### Formulations:

Formulation 1: 40% propylene glycol, 0.7% NaCl, 50 mM TRIS, 5 mg/mL methionine, 3 mg/mL m-cresol, 1 mg/mL teriparatide, pH 7.4
Formulation 2: 30% propylene glycol, 0.7% NaCl, 50 mM TRIS, 5 mg/mL methionine, 3 mg/mL m-cresol, 1 mg/mL teriparatide, pH 7.4
Formulation 3: 20% propylene glycol, 0.7% NaCl, 50 mM TRIS, 5 mg/mL methionine, 3 mg/mL m-cresol, 1 mg/mL teriparatide, pH 7.4
Formulation 4: 30% propylene glycol, 0.7% NaCl, 50 mM TRIS, 5 mg/mL methionine, 3 mg/mL m-cresol, 0.4 mg/mL teriparatide, pH 7.4
   Formulation concentrations will be adjusted for the assay result of 90.8% for PTH(1-34).

### Teriparatide Dose Preparation Instructions for Formulations 1-4:

There are 3 steps to dose preparation:
1. Preparation of stock aqueous TRIS buffer
2. Preparation of solvent stock solutions using aqueous TRIS Buffer
3. Preparation of dosing solutions by adding teriparatide to aliquot of solvent stock solutions or diluting dosing solution with aliquot of solvent stock solution
   The instructions for each of these steps are described in detail as follows:
   Preparation of Stock Aqueous TRIS Buffer (125 mM TRIS, 1.75% NaCl, 12.5 mg/mL Methionine, 7.5 mg/mL m-cresol, pH 7.4):
1. Dispense 90 mL WFI (water for injection) in a clean container.
2. Add 1.75 g NaCl.
3. Add 1.51 g Tris base.
4. Add 1.25 g L-methionine.
5. Add 750 mg m-cresol, USP
6. Stir until dissolved and adjust pH with HCl or NaOH to 7.4. Record temperature of room.
7. Adjust volume to 100 mL total with addition of WFI and mix.

### Preparation of Solvent Stock Solutions Using Stock Aqueous TRIS Buffer:

1. Prepare three separate solutions by adding TRIS buffer from above and solvent, according to Table 3-1 below
2. Stir until dissolved.
3. Adjust pH to 7.4 with HCl or NaOH.
4. Adjust weight to 100 g total with addition of WFI and mix.
5. Recheck and record pH.

TABLE 3-1

| | Aqueous TRIS | | |
|---|---|---|---|
| Description | Buffer | Solvent | WFI |
| 40% Propylene Glycol Stock | 40 (+/−1) g | 40 g propylene | QS to |
| | | glycol | 100 g |
| 30% Propylene Glycol Stock | 40 (+/−1) g | 30 g propylene | QS to |
| | | glycol | 100 g |
| 20% Propylene Glycol Stock | 40 (+/−1) g | 20 g propylene | QS to |
| | | glycol | 100 g |

### Preparation of Formulations 1, 2 and 3 Dosing Solutions:

1. Prepare Formulations 1, 2 and 3 dosing solutions by adding teriparatide to aliquot of solvent stock solutions, according to Table 3-2 below, and mix until teriparatide is dissolved (˜5 min).
2. Adjust pH to nominal 7.4 (+/−0.3) with HCl or NaOH (with <3% dilution of formulation).
3. Record room temperature.

TABLE 3-2

| | | Solvent | |
|---|---|---|---|
| Formulation | Description | Solution | teriparatide* |
| 1 | 40% propylene glycol 1 mg/mL | 10 mL | 11 mg |
| | teriparatide | | |
| 2 | 30% propylene glycol 1 mg/mL | 10 mL | 11 mg |
| | teriparatide | | |
| 3 | 20% propylene glycol 1 mg/mL | 10 mL | 11 mg |
| | teriparatide | | |

*Takes into account peptide assay of 90.8%

### Preparation of Group 4 Dosing Solution:

1. Prepare Formulation 4 dosing solution by diluting Formulation 2 dosing solution with aliquot of 30% propylene glycol solvent stock solution, according to Table 3-3 below, and mix.
2. Adjust pH to nominal 7.4 (+/−0.3) with HCl or NaOH (with <3% dilution of formulation) if necessary.
3. Record room temperature.

TABLE 3-3

| | | Group 2 | 30% Propylene glycol |
|---|---|---|---|
| Formulation | Description | solution | Stock solution |
| 4 | 30% Propylene glycol, | 4 mL | 6 mL |
| | 0.4 mg/mL teriparatide | | |

After dosing, remaining dosing formulations 1-4 are stored at −20° C.

### Example 4

### Subcutaneous Formulations of PTH

Formulations are prepared by repeating the steps of Example 20 wherein a polyol is added, the preservative is benzyl alcohol and the pH adjusted to 7.1. The following formulations are produced:
Formulation 1: 40% propylene glycol 50 mM TRIS, 5 mg/mL methionine, 100 mg/ml sucrose, 0.9% benzyl alcohol, 0.5 mg/mL teriparatide, pH 7.1.
Formulation 2: 30% propylene glycol 50 mM TRIS, 5 mg/mL methionine, 100 mg/ml sucrose, 0.9% benzyl alcohol 0.5 mg/mL teriparatide, pH 7.1.
Formulation 3: 20% propylene glycol 50 mM TRIS, 5 mg/mL methionine, 100 mg/ml sucrose, 0.9% benzyl alcohol 0.5 mg/mL teriparatide, pH 7.1.

### Example 5

### Subcutaneous Formulations of PTH

Formulations are prepared by repeating the steps of Example 20 wherein a polyol is added, the preservative is benzyl alcohol and the pH adjusted to 4.5. The following formulations are produced:
Formulation 1: 40% propylene glycol, 10 mM sodium acetate, 5 mg/mL methionine, 100 mg/ml sucrose, 0.9% benzyl alcohol, 0.5 mg/mL teriparatide, pH 4.5.
Formulation 2: 30% propylene glycol, 10 mM sodium acetate, 5 mg/mL methionine, 100 mg/ml sucrose, 0.9% benzyl alcohol, 0.5 mg/mL teriparatide, pH 4.5.
Formulation 3: 20% propylene glycol, 10 mM sodium acetate, 5 mg/mL methionine, 100 mg/ml sucrose, 0.9% benzyl alcohol, 0.5 mg/mL teriparatide, pH 4.5.

### Example 6

### Subcutaneous Formulations of PTH

Formulations are prepared by repeating the steps of Example 20 wherein diethylene glycol monoethyl ether (DEGMEE) is used instead of propylene glycol. The following formulations are produced:
Formulation 1: 40% 1,2-ethanediol, 0.7% NaCl, 50 mM TRIS, 5 mg/mL methionine, 3 mg/mL m-cresol, 1 mg/mL teriparatide, pH 7.4.
Formulation 2: 30% 1,2-ethanediol, 0.7% NaCl, 50 mM TRIS, 5 mg/mL methionine, 3 mg/mL m-cresol, 1 mg/mL teriparatide, pH 7.4.
Formulation 3: 20% 1,2-ethanediol, 0.7% NaCl, 50 mM TRIS, 5 mg/mL methionine, 3 mg/mL m-cresol, 1 mg/mL teriparatide, pH 7.4.
Formulation 4: 30% 1,2-ethanediol, 0.7% NaCl, 50 mM TRIS, 5 mg/mL methionine, 3 mg/mL m-cresol, 0.4 mg/mL teriparatide, pH 7.4.

### Example 7

### Subcutaneous Formulations of Insulin

Formulations are prepared by repeating the steps of Example 20 wherein insulin is used instead of hPTH. The following formulations are produced:
Formulation 1: 40% propylene glycol, 0.7% NaCl, 50 mM TRIS, 3 mg/mL m-cresol, 1 mg/mL insulin, pH 7.4.
Formulation 2: 30% propylene glycol, 0.7% NaCl, 50 mM TRIS, 3 mg/mL m-cresol, 1 mg/mL insulin, pH 7.4.
Formulation 3: 20% propylene glycol, 0.7% NaCl, 50 mM TRIS, 3 mg/mL m-cresol, 1 mg/mL insulin, pH 7.4.
Formulation 4: 30% propylene glycol, 0.7% NaCl, 50 mM TRIS, 3 mg/mL m-cresol, 0.4 mg/mL insulin, pH 7.4.

### Example 8

### Intravenous or Subcutaneous Formulations of Calcitonin

Formulations are prepared by repeating the steps of Example 20 wherein calcitonin is used instead of hPTH The following formulations are produced:
Formulation 1: 40% propylene glycol, 0.7% NaCl, 50 mM TRIS, 3 mg/mL m-cresol, 1 mg/mL calcitonin, pH 7.1.
Formulation 1: 40% diethylene glycol monoethyl ether, 50 mM TRIS, 5 mg/mL methionine, 100 mg/ml sucrose, 0.9% benzyl alcohol, 0.5 mg/mL calcitonin, pH 7.1.
Formulation 2: 30% diethylene glycol monoethyl ether 50 mM TRIS, 5 mg/mL methionine, 100 mg/ml sucrose, 0.9% benzyl alcohol 0.5 mg/mL calcitonin, pH 7.1.
Formulation 3: 20% diethylene glycol monoethyl ether 50 mM TRIS, 5 mg/mL methionine, 100 mg/ml sucrose, 0.9% benzyl alcohol 0.5 mg/mL calcitonin, pH 7.1.

### Example 9

### Subcutaneous Formulations of PTH

Formulations are prepared by repeating the steps of Example 20 wherein a polyol is added, the preservative is benzyl alcohol and the pH adjusted to 4.5. The following formulations are produced:
Formulation 1: 40% diethylene glycol monoethyl ether, 10 mM sodium acetate, 5 mg/mL methionine, 100 mg/ml sucrose, 0.9% benzyl alcohol, 0.5 mg/mL teriparatide, pH 4.5.
Formulation 2: 30% diethylene glycol monoethyl ether, 10 mM sodium acetate, 5 mg/mL methionine, 100 mg/ml sucrose, 0.9% benzyl alcohol, 0.5 mg/mL teriparatide, pH 4.5.
Formulation 3: 20% diethylene glycol monoethyl ether, 10 mM sodium acetate, 5 mg/mL methionine, 100 mg/ml sucrose, 0.9% benzyl alcohol, 0.5 mg/mL teriparatide, pH 4.5.

### Example 10

### Intravenous or Subcutaneous Formulations of Enbrel

Formulations are prepared by repeating the steps of Example 20 wherein etanercept is used instead of hPTH The following formulations are produced:
Formulation 1: 40% propylene glycol, 100 mg/ml sucrose, 50 mM TRIS, 0.9% benzyl alcohol, 25 mg/mL etanercept, pH 7.4.
Formulation 2: 30% propylene glycol, 100 mg/ml sucrose, 50 mM TRIS, 0.9% benzyl alcohol, 25 mg/mL etanercept, pH 7.4.
Formulation 3: 20% propylene glycol, 100 mg/ml sucrose, 50 mM TRIS, 0.9% benzyl alcohol, 25 mg/mL etanercept, pH 7.4.
Formulation 4: 20% propylene glycol, 100 mg/ml sucrose, 50 mM TRIS, 0.9% benzyl alcohol, 50 mg/mL etanercept, pH 7.4.

### Example 11

### Intravenous or Subcutaneous Formulations of Raptiva

Formulations are prepared by repeating the steps of Example 20 wherein efalizumab is used instead of hPTH. The following formulations are produced:
Formulation 1: 40% propylene glycol, 0.7% NaCl, 50 mM TRIS, 3 mg/mL m-cresol, 100 mg/mL efalizumab, pH 7.4.
Formulation 2: 30% propylene glycol, 0.7% NaCl, 50 mM TRIS, 3 mg/mL m-cresol, 100 mg/mL efalizumab, pH 7.4.
Formulation 3: 20% propylene glycol, 0.7% NaCl, 50 mM TRIS, 3 mg/mL m-cresol, 100 mg/mL efalizumab, pH 7.4.
Formulation 4: 30% propylene glycol, 0.7% NaCl, 50 mM TRIS, 3 mg/mL m-cresol, 40 mg/mL efalizumab, pH 7.4.

### Example 12

### Subcutaneous Formulations of Raptiva

Formulations are prepared by repeating the steps of Example 24 wherein a polyol is added, the preservative is benzyl alcohol and the pH adjusted to 6.2. The following formulations are produced:
Formulation 1: 40% diethylene glycol monoethyl ether, 50 mM histidine, 100 mg/ml sucrose, 0.9% benzyl alcohol, 100 mg/ml efalizumab, pH 6.2.
Formulation 2: 30% diethylene glycol monoethyl ether, 50 mM histidine, 100 mg/ml sucrose, 0.9% benzyl alcohol, 100 mg/ml efalizumab, pH 6.2.
Formulation 3: 20% diethylene glycol monoethyl ether, 50 mM histidine, 100 mg/ml sucrose, 0.9% benzyl alcohol, 100 mg/ml efalizumab, pH 6.2.

### Example 13

### Intravenous or Subcutaneous Formulations of Glucagon

Formulations are prepared by repeating the steps of Example 20 wherein glucagon is used instead of hPTH The following formulations are produced:
Formulation 1: 40% propylene glycol, 0.7% NaCl, 50 mM TRIS, 3 mg/mL m-cresol, 10 mg/mL glucagon, pH 7.4.
Formulation 2: 30% propylene glycol, 0.7% NaCl, 50 mM TRIS, 3 mg/mL m-cresol, 10 mg/mL glucagon, pH 7.4.
Formulation 3: 20% propylene glycol, 0.7% NaCl, 50 mM TRIS, 3 mg/mL m-cresol, 10 mg/mL glucagon, pH 7.4.
Formulation 4: 30% propylene glycol, 0.7% NaCl, 50 mM TRIS, 3 mg/mL m-cresol, 5 mg/mL glucagon, pH 7.4.

### Example 14

### A Randomized, Single-Blind, 5-Period Crossover Study to Evaluate the Pharmacokinetics and Tolerability of Two Subcutaneous Formulations of Teriparatide Compared to Forteo (Teriparatide) and Intravenous Teriparatide Following Single-Dose Administration in Healthy Adult Subjects

Teriparatide 20 μg is the currently marketed dose for all indications. Preclinical data suggest that teriparatide formulated in propylene glycol (PG) may provide improved bioavailability and stability relative to Forteo®. A dose range of teriparatide from 15 μg to 25 μg was selected in order to obtain relative bioavailability data. The 15 μg dose of IV teriparatide was selected to provide a 1:1 absolute bioavailability comparison with 2 of the teriparatide SC injections. This dose is less than the dose that was administered in the absolute bioavailability study conducted for teriparatide (17.5 μg), as documented in the Food and Drug Administration (FDA) approval package notes of Forteo®.

### Methodology:

This was a Phase I, single-center, single-dose, single-blind, randomized, 5-period crossover study in healthy subjects to evaluate the PK and tolerability of 2 SC formulations of teriparatide compared to teriparatide and IV teriparatide following single dose administration. Five treatment regimens (A, B, C, D, and E) were administered to each subject during Periods 1 through 4 in a sequence assigned by randomization. Regimen E was administered in Period 5. Table 14-1 provides a description of each treatment regimen:

TABLE 14-1
Treatments Administered

| Regimen | Description |
|---|---|
| A | teriparatide 15 μg SC (Formulation containing 20% PG; |
| | volume 30 μL) |
| B | teriparatide 15 μg SC (Formulation containing 30% PG; |
| | volume 30 μL) |
| C | teriparatide 25 μg SC (Formulation containing 30% PG; |
| | volume 50 μL) |
| D | Forteo ® 20 μg SC |
| E | teriparatide 15 μg IV |

PG = propylene glycol;
SC = subcutaneous;
IV = intravenous.

### Subcutaneous Dose of Teriparatide (Regimens A, B, and C)

teriparatide was formulated as a clear and colorless sterile solution containing PG (concentration varies), Tris 50 mM, L-methionine 5 mg/mL, sucrose 100 mg/mL, and benzyl alcohol 0.9% which was injected subcutaneously into the abdominal wall following measurement of the appropriate volume for a given dose. Regimen A was formulated with 20% PG and given as 15 μg in 30 μL. Regimens B and C were formulated with 30% PG. Regimen B was given as 15 μg in 30 μL, while Regimen C was given as 25 μg in 50 μL.

Subcutaneous Dose of Teriparatide (Forteo®) (Regimen D)

Teriparatide (Forteo®) is formulated as a clear and colorless sterile isotonic solution in a glass cartridge which was pre-assembled into a disposable delivery device (pen) for SC injection. Each mL of solution contains 250 μg teriparatide (corrected for acetate, chloride, and water content), 0.41 mg glacial acetic acid, 0.1 mg sodium acetate (anhydrous), 45.4 mg mannitol, 3 mg Metacresol, and water for injection. In addition, hydrochloric acid solution 10% and/or sodium hydroxide solution 10% may have been added to adjust the product to pH 4. Each cartridge, pre-assembled into a delivery device, delivers 20 μg of teriparatide per dose each day for up to 28 days. For this study, all subjects received the 20 μg (in 80 μl) injection in the abdominal wall for consistency.

Teriparatide was manufactured by Lilly France—F-67640 Fegersheim, France for Eli Lilly and Company—Indianapolis, Ind., USA. It was supplied in a pre-filled pen delivery device, NDC 0002-8400-01 (MS8400).

Intravenous Dose of Teriparatide (Regimen E)

teriparatide was formulated as a clear and colorless sterile solution containing mannitol 45.4 mg/mL, Na⁺ acetate (anhydrous) 0.1 mg/mL, and acetic acid 0.41 mg/mL. It was injected intravenously using an angiocath in a volume of 60 μL (containing 15 μg). The angiocath was primed with 1 ml of sterile saline (0.9% NaCl, preservative-free) and following administration of teriparatide flushed with 2 ml of sterile saline (0.9% NaCl, preservative-free) in order to ensure that all the teriparatide reaches the blood stream.

Pharmacokinetics

Summaries and analyses of PK results were performed using the PK Evaluable Population, which consisted of all subjects from the Safety Population who had adequate and reliable PK data. The PK parameters were calculated for each subject with sufficient plasma teriparatide or teriparatide concentration data for analysis using the standard non compartmental method.

Plasma PK parameters were tabulated by treatment regimen and summary statistics, including number of subjects, arithmetic mean, standard deviation (SD), minimum, maximum, median, coefficients of variation (CV %), and geometric mean. The upper and lower limits of the 90% confidence intervals (CI) were presented for Cₘₐₓ, t_{1/2}, AUCs, CL/F, and V_{z}/F. In addition, the geometric mean and CV % were calculated for Cₘₐₓ and AUC values.

Bioavailability

A mixed model was used for the analysis of bioavailability on the logarithm-transformed PK parameters (AUC₀₋ₜ, AUC_{0-inf}, and Cₘₐₓ). The mixed model included treatment (formulation), period and sequence as fixed factors and subject within sequence as random effect. A 10% level of significance was used to test the sequence effect.

Pharmacokinetic parameters (AUC₀₋ₜ, AUC_{0-inf}, and Cₘₐₓ) of SC teriparatide doses and teriparatide were used for the bioavailability analyses (relative, absolute, and apparent absolute). The PK parameters were logarithm-transformed for statistical analyses. The least-square (LS) means, the difference between LS means of the different drug formulations, and the standard error associated with the difference were calculated with the mixed model. These values were back-transformed to the original scale. The bioavailability was evaluated by calculating a 90% CI of the difference between LS mean (expressed as a percentage relative to the reference formulation).

Summary of Results:

Pharmacokinetic/Pharmacodynamic:

The geometric mean for AUC₀₋ₜ for PTH (1-34) concentration was 126.98 hr*pg/mL for treatment A, 103.20 hr*pg/mL for treatment B, 183.84 hr*pg/mL for treatment C, 80.44 hr*pg/mL for treatment D, and 141.60 hr*pg/mL for treatment E.

The mean AUC₀₋ₜ for PTH (1-34) concentration was 133.27 hr*pg/mL for treatment A, 110.34 hr*pg/mL for treatment B, 188.81 hr*pg/mL for treatment C, 88.93 hr*pg/mL for treatment D, and 147.82 hr*pg/mL for treatment E.

The absolute bioavailability analysis (test formulations vs. IV formulation) demonstrated the following for dn-AUC₀₋ₜ:

Ratio of geometric LS means of 0.877 (CI 0.751, 1.024) for treatment A

Ratio of geometric LS means of 0.722 (CI 0.618, 0.843) for treatment B

Ratio of geometric LS means of 0.771 (CI 0.660, 0.901) for treatment C

The relative bioavailability analysis (test formulations vs. teriparatide) demonstrated the following for dn-AUC₀₋ₜ:

Ratio of geometric LS means of 2.078 (CI 1.785, 2.419) for treatment A

Ratio of geometric LS means of 1.711 (CI 1.469, 1.992) for treatment B

Ratio of geometric LS means of 1.828 (CI 1.570, 2.129) for treatment C

The apparent absolute bioavailability analysis (teriparatide vs. IV formulation) yielded a ratio of the geometric LS mean for dn-AUC₀₋ₜ of 0.422 (CI 0.361, 0.493). Pharmacokinetic and Pharmacodynamic Results

Table 14-3 presents the PK results for each treatment for the PK Evaluable Population. The mean AUC₀₋ₜ was 133.27 hr*pg/mL (SD 39.87), for treatment A, 110.34 hr*pg/mL (SD 40.19) for treatment B, 188.81 hr*pg/mL (SD 46.01) for treatment C, 88.93 (SD 35.89) for treatment D, and 147.82 (SD 38.35) for treatment E.

TABLE 14-3
PHARMACOKINETIC PARAMETERS FOR
PLASMA PTH (1-34) - PK EVALUABLE POPULATION

| Treatment Regimen: | | | | |
|---|---|---|---|---|
| A | B | C | | |
| Teriperatide | Teriperatide | Teriperatide | | |
| 15 μg SC | 15 μg SC | 25 μg SC | D | E |
| (20% | (30% | (30% | Forteo ® | Teriperatide |
| PG) | PG) | PG) | 20 μg SC | 15 μg IV |

AUC₀₋₈ₕᵣ (hr * pg/mL)

| | | | | | | |
|---|---|---|---|---|---|---|
| All | Arith. Mean, | 133.27, | 110.34, | 188.81, | 88.93, | 147.82, |
| Subjects | SD | 39.87 | 40.19 | 46.01 | 35.89 | 38.35 |
| | CV % | 29.91 | 36.42 | 24.37 | 40.36 | 25.94 |
| | Geom. Mean | 126.98 | 103.20 | 183.84 | 80.44 | 141.60 |

AUC₀₋ₜ (hr * pg/mL)

| | | | | | | |
|---|---|---|---|---|---|---|
| All | Arith. Mean, | 133.27, | 110.34, | 188.81, | 88.93, | 147.82, |
| Subjects | SD | 39.87 | 40.19 | 46.01 | 35.89 | 38.35 |
| | CV % | 29.91 | 36.42 | 24.37 | 40.36 | 25.94 |
| | Geom. Mean | 126.98 | 103.20 | 183.84 | 80.44 | 141.60 |

AUC_{0-inf} (hr * pg/mL)

| | | | | | | |
|---|---|---|---|---|---|---|
| All | Arith. Mean, | 197.90, | 204.10, | 248.57, | 125.48, | 151.99, |
| Subjects | SD | 45.98 | 49.47 | 57.82 | 26.70 | 39.27 |
| | CV % | 23.23 | 24.24 | 23.26 | 21.28 | 25.84 |
| | Geom. Mean | 193.34 | 200.00 | 242.20 | 122.26 | 145.71 |

Cₘₐₓ (pg/mL)

| | | | | | | |
|---|---|---|---|---|---|---|
| All | Arith. Mean, | 64.58, | 59.49, | 78.43, | 76.87, | 563.43, |
| Subjects | SD | 25.03 | 24.71 | 26.33 | 34.94 | 148.73 |
| | CV % | 38.76 | 41.53 | 33.57 | 45.45 | 26.40 |
| | Geom. Mean | 59.25 | 54.29 | 74.92 | 69.96 | 540.38 |

tₘₐₓ (h)

| | | | | | | |
|---|---|---|---|---|---|---|
| All | Median (Min., | 1.00 | 1.00 | 1.03 | 0.25 | 0.08 |
| Subjects | Max.) | (0.25, | (0.50, | (0.25, | (0.25, | (0.08, |
| | | 3.00) | 2.00) | 2.00) | 0.55) | 0.08) |

t_{1/2} (h)

| | | | | | | |
|---|---|---|---|---|---|---|
| All | Arith. Mean, | 1.54, | 1.99, | 1.37, | 0.86, | 0.10, |
| Subjects | SD | 1.39 | 1.27 | 0.52 | 0.28 | 0.03 |
| | CV % | 90.36 | 63.91 | 38.16 | 32.88 | 28.74 |
| | Geom. Mean | 1.18 | 1.70 | 1.28 | 0.82 | 0.10 |

Cl/F (L/hr)

| | | | | | | |
|---|---|---|---|---|---|---|
| All | Arith. Mean, | 79.32, | 76.40, | 106.14, | 169.17, | 109.35, |
| Subjects | SD | 17.11 | 16.11 | 27.43 | 52.79 | 47.88 |
| | CV % | 21.57 | 21.09 | 25.85 | 31.21 | 43.79 |
| | Geom. Mean | 77.58 | 75.00 | 103.22 | 163.59 | 102.94 |

Vz/F (L)

| | | | | | | |
|---|---|---|---|---|---|---|
| All | Arith. Mean, | 158.13, | 226.55, | 208.22, | 215.27, | 15.57, |
| Subjects | SD | 107.64 | 171.16 | 90.73 | 103.13 | 5.35 |
| | CV % | 68.07 | 75.55 | 43.58 | 47.91 | 34.37 |
| | Geom. Mean | 131.61 | 184.47 | 190.61 | 193.43 | 14.86 |

CV % = coefficients of variance;
IV = intravenous;
PG = propylene glycol;
SD = standard deviation.

### Bioavailability

A comparison of formulations was conducted using geometric LS Means. The values for AUC₀₋ₜ, AUC_{0-inf} and Cₘₐₓ were dose-normalized (dn) for both the test formulations and the reference formulations.

### Relative Bioavailability

The ratios of the geometric LS means of dn-AUC₀₋ₜ are summarized below.

TABLE 14-2
Ratio of Geometric LS Means for Treatments A, B and C
versus Teriparatide

| | Ratio of Geom LS | |
|---|---|---|
| Treatment Regimen | Means | 90% CI for Ratio |
| A - teriparatide 15 μg SC | 2.078 | (1.785, 2.419) |
| (20% PG) | | |
| B - teriparatide 15 μg SC (30% | 1.711 | (1.469, 1.992) |
| PG) | | |
| C - teriparatide 25 μg SC (30% | 1.828 | (1.570, 2.129) |
| PG) | | |

PG = propylene glycol;
SC = subcutaneous.

The ratios of the geometric LS means for dn-AUC_{0-inf} for treatment regimens A, B and C versus teriparatide were 2.064, 2.128 and 1.645, respectively. The ratios of the geometric LS means for dn-Cₘₐₓ for treatment regimens A, B and C versus teriparatide were 1.099, 1.035 and 0.857, respectively.

What is claimed is:

1. A pharmaceutically acceptable parenteral composition comprising:
(a) a pharmaceutically effective amount of a human parathyroid hormone (PTH) or active fragment, variant, or mimetic thereof; and
(b) a bioavailability enhancer in an amount sufficient to increase the bioavailability of said PTH or active fragment, variant, or mimetic thereof, wherein the bioavailability enhancer is a diol or diol ether,
wherein the diol is selected from the group consisting of: 1,2-ethanediol; 1,2-propanediol; 1,3-propanediol; 1,2-butanediol; 1,3-butanediol; 2,3-butanediol; 1,4-butanediol; but-2-ene-1,4-diol; 1,2-pentanediol; 1,4-Pentanediol, 2,4-Pentanediol, 1,5-pentanediol; 1,2-hexanediol, 1,5-Hexanediol, 2,5-Hexanediol; 1,6-hexanediol; 1,2-Heptanediol; 1,7-Heptanediol; 1,2-Octanediol; 1,8-Octanediol; 1,2-Nonanediol; 1,9-Nonanediol; 1,2-Decanediol; 1,10-decanediol; 1,2-dodecanediol; 1,12-dodecanediol; 3-methylpentane-1,5-diol; 2,5-dimethyl-1,3-hexanediol; 1,2-cyclopentanediol, 2,2,4-trimethyl-1,3-pentanediol; 1,2-cyclohexanediol; 1,4-cyclohexanediol; 1,4-bis(hydroxymethyl)cyclohexane; neopentyl glycol hydroxypivalate; 2,2-bis(4-hydroxyphenyl)propane; 2,2-bis[4-(2-hydroxypropyl)phenyl]propane; diethylene glycol; triethylene glycol; tetraethylene glycol; dipropylene glycol; tripropylene glycol; tetrapropylene glycol; and 3-thiopentane-1,5-diol, 2,2-Dimethyl-1,3-propanediol 2,3-Dimethyl-2,3-Butanediol; 2,3-Dimethyl-1,2-Butanediol, 1-Phenyl-1,2-Propanediol; and 2-Methyl-1,3-Propanediol;
and wherein the diol ether is a diol ether according to the structure:
R—(OCH₂CH₂)n-OR′
where: n=1, 2, or 3; R=alkyl C7 or less or R=phenyl or alkyl substituted phenyl; R′=H or alkyl C7 or less or OR′ is a carboxylic acid ester, sulfate, phosphate, nitrate, or sulfonate;
or the diol ether is a diol ether according to the structure:
R—[OCH₂CH(CH₃)]n-OR′
where: n=1, 2, or 3; R=alkyl C7 or less or R=phenyl or alkyl substituted phenyl; R′=H or alkyl C7 or less or OR′ is a carboxylic acid ester, sulfate, phosphate, nitrate, or sulfonate.

2. The composition of claim 1, wherein said human parathyroid hormone is a PTH variant selected from the group consisting of PTH(1-31), PTH(1-34), PTH(1-37), PTH(1-38), PTH(1-41) and PTH(1-84).

3. The composition according to claim 1, wherein the peptide variant is PTH(1-34).

4. The composition of claim 1, wherein the PTH variant is cyclic.

5. The composition of claim 1, wherein the composition increases the blood levels of the peptide or peptide mimetic to greater than 10 to 20% AUC when administered to a subject as compared to a composition that is absent the bioavailability enhancer.

6. The composition of claim 1, wherein the composition increases the blood levels of the peptide or peptide mimetic to greater than 10% Cmax when administered to a subject as compared to a composition that is absent the bioavailability enhancer.

7. The composition of claim 1, wherein the bioavailability enhancer is present at concentration from about 10% to about 60%.

8. The composition of claim 1, further comprising a antimicrobial preservative.

9. The composition of claim 1, further comprising m-cresol, benzalkonium chloride or chloroethanol, parabens, or benzyl alcohol.

10. The composition of claim 1, wherein the diol or diol ether is propylene glycol.

11. The composition of claim 1, wherein the bioavailability enhancer is diethylene glycol monoethylether.

12. A composition according to claim 1 in a sealed sterile glass vial.

13. The composition according to claim 1, wherein the composition is formulated for subcutaneous administration.

14. A method of treating a subject in need thereof comprising administering to the subject the parenteral composition of claim 1 wherein the amount of bioavailability enhancer is sufficient to reduce the dose given to achieve the same pharmacological activity of said PTH when administered to a subject as compared to a composition that is absent the bioavailability enhancer.

15. A method of reducing the side effects associated with treating a subject with PTH comprising administering to the subject the parenteral composition of claim 1 wherein the amount of bioavailability enhancer is sufficient to reduce the dose given to achieve the same pharmacological activity of said PTH when administered to a subject as compared to a composition that is absent the bioavailability enhancer.

16. A kit comprising
(a) a pharmaceutically effective amount of a human parathyroid hormone (PTH) or active fragment, variant, or mimetic thereof; and
(b) a solution for a reconstituting the lyophilized peptide or peptide mimetic comprising a bioavailability enhancer in an amount sufficient to increase the bioavailability of said PTH or active fragment, variant, or mimetic thereof, wherein the bioavailability enhancer is a diol or diol ether.
wherein the diol is selected from the group consisting of: 1,2-ethanediol; 1,2-propanediol; 1,3-propanediol; 1,2-butanediol; 1,3-butanediol; 2,3-butanediol; 1,4-butanediol; but-2-ene-1,4-diol; 1,2-pentanediol; 1,4-Pentanediol, 2,4-Pentanediol, 1,5-pentanediol; 1,2-hexanediol, 1,5-Hexanediol, 2,5-Hexanediol; 1,6-hexanediol; 1,2-Heptanediol; 1,7-Heptanediol; 1,2-Octanediol; 1,8-Octanediol; 1,2-Nonanediol; 1,9-Nonanediol; 1,2-Decanediol; 1,10-decanediol; 1,2-dodecanediol; 1,12-dodecanediol; 3-methylpentane-1,5-diol; 2,5-dimethyl-1,3-hexanediol; 1,2-cyclopentanediol, 2,2,4-trimethyl-1,3-pentanediol; 1,2-cyclohexanediol; 1,4-cyclohexanediol; 1,4-bis(hydroxymethyl)cyclohexane; neopentyl glycol hydroxypivalate; 2,2-bis(4-hydroxyphenyl)propane; 2,2-bis[4-(2-hydroxypropyl)phenyl]propane; diethylene glycol; triethylene glycol; tetraethylene glycol; dipropylene glycol; tripropylene glycol; tetrapropylene glycol; and 3-thiopentane-1,5-diol, 2,2-Dimethyl-1,3-propanediol 2,3-Dimethyl-2,3-Butanediol; 2,3-Dimethyl-1,2-Butanediol, 1-Phenyl-1,2-Propanediol; and 2-Methyl-1,3-Propanediol;
and wherein the diol ether is a diol ether according to the structure:
R—(OCH₂CH₂)n-OR′
where: n=1, 2, or 3; R=alkyl C7 or less or R=phenyl or alkyl substituted phenyl; R′=H or alkyl C7 or less or OR′ is a carboxylic acid ester, sulfate, phosphate, nitrate, or sulfonate;
or the diol ether is a diol ether according to the structure:
R—[OCH₂CH(CH₃)]n-OR′
where: n=1, 2, or 3; R=alkyl C7 or less or R=phenyl or alkyl substituted phenyl; R′=H or alkyl C7 or less or OR′ is a carboxylic acid ester, sulfate, phosphate, nitrate, or sulfonate.
